(19) United States
(12) Patent Application Publication
(10) Pub. No.: US 2007/0275872 A1
(43) Pub. Date: Nov. 29, 2007

(54) Peptides with Anti-Obesity Activity and Other Related Uses

(76) Inventors: Garth J.S. Cooper, Auckland (NZ); Yu Wang, Hong Kong (CN); Aimin Xu, Hong Kong (CN)

Correspondence Address: DUANE MORRIS LLP, 101 WEST BROADWAY, SUITE 900, SAN DIEGO, CA, 92101-8285, US

(21) Appl. No.: 10/577,792

(22) PCT Filed: Oct. 28, 2004
(86) PCT No.: PCT/NZ04/00271

§ 371 Date: Apr. 2, 2007

(30) Foreign Application Priority Data
Oct. 28, 2003 (NZ) 529220

(51) Int. Cl. A61K 38/00; A61P 43/00; C07H 21/04; C07K 14/00; C12N 1/00; C12N 15/00; C12N 5/00

(52) U.S. Cl. 514/2; 435/252.3; 435/320.1; 435/325; 530/300; 536/23.5

(57) ABSTRACT

Novel peptides and uses thereof, including polypeptides and related molecules with uses, for example, in weight loss and for the treatment of obesity and related conditions associated with, for example, increased mass of adipocytes.

### FIELD

Novel peptides and uses thereof, including polypeptides and related molecules with uses, for example, in weight loss and for the treatment of obesity and related conditions associated with, for example, increased mass of adipocytes.

### BACKGROUND

The following description includes information that may be useful in understanding the present invention. It is not an admission that any of the information provided herein is prior art, or relevant, to the presently described or claimed inventions, or is a reference that may be used in evaluating patentability.

Insulin resistance, characterized by diminished sensitivity of insulin in its target tissues, is a fundamental aspect of the aetiology of type 2 diabetes and is often associated with other diseases such as, for example, hyperlipidemia, atherosclerosis and hypertension (i.e., syndrome X). The molecular basis of insulin resistance is complex and multifactorial. A close correlation is reported to exist between changes in fat mass and insulin sensitivity. Altered functions of adipocytes may play an important role in this process. Insulin resistance and hyperinsulinaemia is reported to occur in obese as well as in lipodystrophic individuals. Two recent independent genetic studies reported that fat-free mice had severe insulin resistance and hyperglycemia

Adipose tissue serves as an energy storage depot for triglycerides. It is also reported to be an active endocrine organ that can secrete a variety of biologically active molecules in response to extracellular signals. Adipocyte-secreted products have been reported to play roles in the regulation of systematic energy homeostasis, and their altered expression and/or secretion may contribute to insulin resistance and its associated syndromes. One adipocyte-secreted product is leptin, which is reported to be a central regulator of adiposity and also affects glucose homeostasis. Other adipocyte-secreted molecules, include TNFα, free fatty acids, and the recently characterized resistin. TNFα has been reported to be overproduced by adipose tissue in insulin resistant states. Increased expression and secretion of plasminogen activator inhibitor 1 (PAI-1) and angiotensinogen in adipose tissue may play a role in obesity and thrombotic vascular disease and hypertension.

A role for adipose tissue as an endocrine organ is also reflected in the recent discovery of adiponectin, a hormone exclusively secreted from adipocytes. It is reported that messenger RNA (mRNA) expression and the secretion level of adiponectin are decreased in a variety of animal models with insulin resistance, as well as in obese humans and type 2 diabetic patients from different ethnic groups. Further, it is reported that replenishment of adiponectin, a purported insulin sensitizer, can decrease hyperglycemia, restore insulin sensitivity and cause sustained weight loss in mice without affecting food intake.

Despite the identification of numerous secretory factors, there may be other yet-unidentified factors that play important roles in modulating energy metabolism. Such a factor, which we discovered and have named, “adipocyspin,” is disclosed and claimed herein.

### BRIEF SUMMARY

The inventions described and claimed herein have many attributes and embodiments including, but not limited to, those set forth or described or referenced in this Brief Summary.

The inventions described and claimed herein are not limited to or by the features or embodiments identified in this Brief Summary, which is included for purposes of illustration only and not restriction.

In one aspect, an isolated polynucleotide is provided that encodes, or is complementary to a sequence that encodes, an adipocyspin polypeptide.

The invention includes, for example, polynucleotides comprising a sequence encoding a polypeptide that has an anti-obesity activity, and active or immunologically active fragments thereof, and which include, for example: (a) a polynucleotide encoding a polypeptide having the amino acid sequence:

[SEQ ID NO: 1]

MKCLLISLALWLGTVGTRGTEPELSETQRRSLQVALEEFHKHPPVQLAFQ
EIGVDRAEEVLFSAGTFVRLEFKLQQTNCPKKDWKKPECTIKPNGRRRKC
LACIKMDPKGKILGRIVHCPILKQGPQDPQELQCIKIAQAGEDPHGYFLP
GQFAFSRALRTK;

(b) a polynucleotide encoding a polypeptide having the amino acid sequence:

[SEQ ID NO: 2]

MRRLLIPLALWLGAVGVGVAELTEAQRRGLQVALEEFHKHPPVQWAFQET
SVESAVDTPFPAGIFVRLEFKLQQTSCRKRDWKKPECKVRPNGRKRKCLA
CIKLGSEDKVLGRLVHCPIETQVLREAEEHQETQCLRVQRAGEDPHSFYF
PGGFAFSKALPRS;

(c) polynucleotides that hybridize under stringent conditions, for example, to a polynucleotide of (a) or (b) or a complement of either thereof; and (d) polynucleotide sequences that are degenerate as a result of the genetic code to the sequences defined in (a), (b) or (c).

In some embodiments the polynucleotide has at least about 10, 15, 25, 50 or 100 contiguous bases identical or exactly complementary to the polynucleotide of (a) or (b).

In other embodiments, the polynucleotide is the full-length sequence of SEQ ID NO:5 or 6 or encodes an adipocyspin polypeptide having the sequence of SEQ ID NO:1 or SEQ ID NO:2 or a fragment of either thereof, including bioactive and immunologically active peptides.

The polynucleotide may be operably linked to a promoter or other sequence that allows or enhances expression of the polynucleotide in a cell, such as an adipocyte or a 3T3 L1 cell, for example.

In another embodiment, a recombinant vector (e.g., an expression vector) is provided for expressing an adipocyspin polypeptide or fragment.

Also provided is a cell (e.g., a bacterial, eukaryotic, mammalian, or human cell) comprising a recombinant adipocyspin polynucleotide or vector, and a method for producing an adipocyspin protein, peptide, or fusion protein or fragment by culturing a cell containing the recombinant adipocyspin polynucleotide or vector coding for an adipocyspin protein, peptide, or fusion protein or fragment under conditions in which the polypeptide may be expressed.

Also provided are isolated, substantially pure, or recombinant adipocyspin polypeptides, or bioactive or immunogenic fragments thereof, including, for example, the polypeptides encoded by (a)-(c) above. In one aspect, for example, the polypeptide has the amino acid sequence identical to SEQ ID NO:1 or SEQ ID NO:2. In another aspect, the polypeptide has an amino acid sequence that differs from SEQ ID NO:1 or SEQ ID NO:2 by conservative mutations, which is at least about 60%, 80%, or 90% or more identical to SEQ ID NO:1 or SEQ ID NO:2, and/or that is immunologically cross-reactive with the full-length polypeptide encoded by SEQ ID NO:1 or SEQ ID NO:2. In other aspects, the polypeptide has one, two or three intramolecular disulphide bonds, for example, polypeptides wherein at least two cysteine residues, for example cysteine residues corresponding to the cysteine residues in human adipocyspin, or at amino acid positions 62, 72, 83, 86, 101 or 116 of mouse adipocyspin, are joined to form an intramolecular disulphide bond. In one aspect, the polypeptide is a fusion protein. In other aspects, for example, the polypeptide has an activity of a naturally occurring human adipocyspin, such as inhibiting the formation of adipocytes from preadipocytes, and/or decreasing body adiposity or adipose tissue mass.

In another embodiment, an antibody, or antibody fragment (e.g., Fab fragment or single chain antibody) or binding fragment (e.g., produced by phage display) that specifically binds to an adipocyspin polypeptide is provided. The antibody may be monoclonal and may bind with an affinity of at least about 10⁸ M⁻¹, preferably, an affinity of at least about 10⁹ M⁻¹ or at least about 10¹¹ M⁻¹. The invention also provides an isolated cell or a hybridoma capable of secreting the antibody, antibody fragment or antibody-binding fragment. The antibody, antibody fragment or antibody-binding fragment may be human or chimeric or humanized.

Also provided is a method of detecting an adipocyspin gene product and/or fragment thereof in a sample by (a) contacting the sample with a probe that specifically binds the gene product and/or fragment thereof, wherein the probe and the gene product and/or fragment thereof form a complex, and detecting formation of the complex; or (b) specifically amplifying the gene product and/or fragment thereof in the biological sample, wherein said gene product and/or fragment thereof is a polynucleotide, and detecting the amplification product; wherein the formation of the complex or presence of the amplification product is correlated with the presence of the adipocyspin gene product and/or fragment thereof in the biological sample. In one embodiment the gene product and/or fragment thereof is a polypeptide and probe is an antibody. In a different embodiment, the gene product and/or fragment thereof is RNA and the probe is a polynucleotide.

In another aspect, a method is provided for identifying a modulator of adipocyspin activity. Such a method may include the steps of (a) contacting a polypeptide, for example an adipocyspin polypeptide, and an adipocyspin receptor and/or an adipocyspin receptor preparation in the presence of a test compound, and (b) comparing the level of binding of the adipocyspin receptor and/or adipocyspin receptor preparation and the polypeptide in (a) with the level of binding in the absence of the test compound, wherein a decrease in binding indicates that the test compound is an inhibitor or blocker of binding and an increase in binding indicates that the test compound is an enhancer or simulator of binding. In one embodiment, a cell expresses the adipocyspin polypeptide.

In another aspect, a method is provided for use in identifying or screening for agonists or antagonists of adipocyspin, which includes bringing together a test sample and an adipocyspin receptor preparation, the test sample containing one or more test compounds, and the adipocyspin receptor preparation containing an adipocyspin receptor protein capable of binding to adipocyspin; incubating the test sample and the receptor preparation under conditions that allow the binding of adipocyspin to the receptor protein; and, identifying those test samples containing one or more test compounds which detectably bind to the receptor protein. In another embodiment, this method further comprises the steps of screening test samples which detectably bind to the receptor protein for in vitro or in vivo stimulation or inhibition of adipocyspin receptor mediated activity, and identifying those test samples which act as agonists or antagonists of adipocyspin. In a preferred embodiment, the test samples which detectably bind to the adipocyspin receptor protein are identified by measuring the displacement of a labeled first ligand from the receptor protein preparation by the test sample, and comparing the measured displacement of the first labeled ligand from the receptor preparation by the test sample with the measured displacement of the labeled first ligand from the receptor preparation by one or more known second ligands. Labeled first ligands and second ligands include adipocyspin, an adipocyspin agonist, or an adipocyspin antagonist. Useful receptor preparations include, for example, isolated cells bearing the adipocyspin receptor, isolated membrane preparations bearing the adipocyspin receptor and isolated adipocyspin receptor protein. When isolated membranes are used as the receptor preparation, especially preferred are membranes from the basal forebrain region. Test samples used in any of the above methods that contain more than one test compound and which yield positive results can then be divided and retested as many times as necessary, and as appropriate, to identify the compound or compounds in the test sample which are responsible for yielding the positive result.

In another aspect, there is provided a method for evaluating one or more receptor binding characteristics sought to be determined for a known or a candidate adipocyspin agonist or antagonist compound, which includes the steps of assessing or measuring the ability of the compound to compete against a labeled ligand for binding to an adipocyspin receptor preparation; and, determining the receptor binding characteristic sought to be determined for said compound. Receptor binding characteristics that may be determined include, for example, binding affinity and binding specificity.

In still another aspect, a method is provided for determining the presence or amount of an adipocyspin receptor binding compound in a test sample to be assayed, which includes the steps of bringing together the test sample and an adipocyspin receptor preparation; measuring the ability of the test sample to compete against a labeled ligand for binding to the adipocyspin receptor preparation; and, optionally, relating the amount of adipocyspin receptor binding compound in the test sample with the amount of adipocyspin receptor binding compound measured for a negative control sample, the negative control sample being known to be free of any adipocyspin receptor binding compound, and/or relating the amount of adipocyspin receptor binding compound in the test sample with the amounts of adipocyspin receptor binding compound measured for positive control samples which contain known amounts of adipocyspin receptor binding compound, in order to determine the presence or amount of adipocyspin receptor binding compound present in the test sample. This assay method, in still further embodiments, can be utilized to evaluate the stability of an adipocyspin preparation, to evaluate the potency of an adipocyspin preparation, and to evaluate the solubility characteristics of an adipocyspin preparation, for example.

In another aspect, receptor preparations of the invention can be utilized to prepare anti-adipocyspin receptor antibodies, including polyclonal antisera and monoclonal antibodies, utilizing art-known methods.

In another aspect, the invention is used to screen cell lines, cells desegregated from tissue, and cells from human or animal blood, for example, in order to identify those which carry adipocyspin receptors. The adipocyspin receptor preparations of the invention may also be bound to a solid phase and used in various affinity chromatography methods and used, for example, for the purification of adipocyspin or the evaluation of samples known or suspected to contain adipocyspin, adipocyspin agonists or adipocyspin antagonists. Also provided is a method of identifying a modulator of adipocyspin activity by, for example, contacting a cell expressing a recombinant adipocyspin polypeptide and a test compound and assaying for a biological effect that occurs in the presence but not absence of the test compound, wherein a test compound that induces a biological effect is identified as a modulator or stimulator of adipocyspin activity. For example, such test compounds may include but are not limited to polynucleotides including those functioning as antisense or RNAi polynucleotides, polypeptides including polypeptides capable of binding to or modifying adipocyspin, and compounds including those capable of modulating the level of adipocyspin capable of exerting a biological effect, for example, binding an adipocyspin receptor. In one embodiment, the biological effect assayed for is the rate of conversion of preadipocytes to adipocytes.

In another aspect, a process is provided for making a pharmaceutical composition by, for example, formulating an adipocyspin or active fragment thereof, or a modulator of adipocyspin activity (e.g., binding), for pharmaceutical use.

In another aspect, pharmaceutical compositions comprising or consisting essentially or, for example, an adipocyspin or active fragment thereof, are provided. Other pharmaceutically acceptable carriers, excipients, etc., may be included

Also provided is a method for identifying compounds useful for the treatment of various diseases or conditions including, for example, adipocyspin-mediated or adipocyspin-involved diseases and conditions, by determining whether the compound interacts with an adipocyspin or an adipocyspin receptor.

A method is also provided for treating subjects for various diseases or conditions including, for example, an adipocyspin-mediated or adipocyspin-involved disease or condition in a mammal by reducing or increasing the activity or expression of adipocyspin in a cell or tissue in the mammal or administering an adipocyspin or an active fragment thereof, or a modulator of adipocyspin, function to the mammal. In various embodiments, the condition or disease is obesity or conditions associated with increased adipose tissue mass. In other embodiments, the mammal wishes to lose weight and/or avoid weight gain.

In another aspect there is provided the use of an adipocyspin or an active fragment thereof, or a modulator of Adipocyspin in the preparation of a medicament for the treatment of various diseases or conditions including for example an Adipocyspin-mediated or Adipocyspin-involved disease or condition in a mammal by reducing or increasing the activity or expression of adipocyspin in a cell or tissue in the mammal. In one embodiment the disease or condition includes, for example, obesity or conditions associated with increased adipose tissue mass, enhancing the effects of insulin. In other embodiments, the mammal wishes to lose weight and/or avoid weight gain.

Also provided is a method for use in determining the activity of an adipocyspin or fragment thereof, which includes bringing together a test sample and an adipocyspin receptor preparation, the test sample containing an adipocyspin or fragment thereof, and the adipocyspin receptor preparation containing an adipocyspin receptor protein capable of binding to an adipocyspin; incubating the test sample and the receptor preparation under conditions that allow the binding of an adipocyspin or fragment thereof to the receptor protein; and, measuring the ability of the test sample to induce a biological effect. In various examples, the adipocyspin receptor preparation comprises or includes preadipocytes such as, for example, 3T3 L1 cells and/or cells(s) expressing an adipocyspin receptor, such as, for example, cells transiently transfected with a polynucleotide encoding an adipocyspin receptor, the biological effect is the conversion of preadipocytes to adipocytes and/or the accumulation of lipid and/or an increase or decrease in the expression of adipocyte markers, such as, for example, PPARγ or GLUT4.

Also provided is a composition comprising an adipocyspin polypeptide wherein the adipocyspin polypeptide is recombinant, isolated, purified, or synthesized. In one embodiment the composition is effective to elicit a plasma adipocyspin polypeptide concentration of between 1 μg/mL and 20 μg/mL, in another embodiment the concentration is between 1.9 μg/mL and 17 μg/mL.

In another aspect a method of diagnosing an individual the presence of, or pre-disposition towards developing, a disease state comprising determining the level of an adipocyspin polypeptide in the individual and comparing the level with a level characteristic of an individual who is not suffering from the disease state, wherein a difference in levels is indicative of the presence of or propensity to develop the disease. In one embodiment the disease state is selected from any one of the following hyperglycemia, insulin resistance, type 2 diabetes mellitus, obesity, hypertension, artherosclerosis, coronary heart disease, ischemic heart disease, polycystic ovary syndrome, and a metabolic syndrome associated with insulin resistance. In a further embodiment the method utilises electrophoresis, HLPC, or mass spectrometry.

Also provided is a method for treating a disease state associated with adipocyspin dysregulation comprising administering an effective amount of a pharmaceutically acceptable composition comprising an adipocyspin polypeptide.

The use of an adipocyspin polypeptide in the manufacture of a medicament, with or without pharmaceutically acceptable excipients, co-actives, diluents and containment vessels, in the preparation of a pharmaceutical composition or medicament or dosage unit useful in a mammal for: i) to treat a disease state associated with adipocyspin polypeptide regulation; or ii) to enhance the effects of insulin; or iii) inhibit obesity or states associated with increased fat mass.

### BRIEF DESCRIPTION OF THE DRAWINGS

FIG. 1 shows 2-dimensional electrophoresis (2-DE) separation of a low molecular weight adipocyte-secreted protein induced during adipose conversion where, after 8 days of induction of differentiation, subconfluent 3T3-L1 preadipocytes or adipocytes were rinsed three times with PBS, and then incubated with serum free DMEM for 4 hr. The medium was collected, concentrated, and 50 μg of proteins from each sample were separated by 2-DE and visualized with silver staining and the protein preferentially secreted in the adipocytes is denoted as the arrow.

FIG. 2 shows microcharacterization of a novel adipocyte-secreted product by reversed phase HPLC and amino acid sequencing. The spots corresponding to the adipocyte-specific protein as from FIG. 1 were excised from multiple Coommassie Brilliant Blue stained gels and subjected to trypsin digestion. The tryptic peptide mixture was separated by RP HPLC to fractionate the peptides and the table shows the amino acid sequences for the indicated RP HPLC fractions.

FIG. 3 shows a sequence analysis of an adipocyspin, where (A) is the amino acid sequence of a mouse adipocyspin, (B) is a schematic diagram of a mouse adipocyspin and (C) is the alignment of cystatin-like domains of a mouse adipocyspin and a mouse cystatin C.

FIG. 4 shows that adipocyspin is secreted from transfected COS 7 cells. COS 7 cells were transiently transfected with a plasmid which encodes COOH terminal FLAG-tagged full-length adipocyspin, and grown in DMEM for 48 hours. The cell culture medium was then collected and concentrated using Vivian concentrator (with molecular weight cut-off (MWCO) of 5000 Da). 20 μg of proteins from either cell pellet or medium were separated by SDS-PAGE and probed with either anti-FLAG monoclonal antibody or anti-β tubulin monoclonal antibody. Adipocyspin can be readily detected in cell medium while β tubulin cannot be detected.

FIG. 5 shows the differentiation-dependent expression of adipocyspin mRNA. Total RNA was purified from NIH 3T3 cells or from 3T3-L1 cells at the indicated time points following hormonal differentiation. 10 μg of total RNA from each sample was subjected to Northern blot analysis using ³²P labelled adipocyspin DNA. The 18 S RNA hybridization signal is shown alongside adipocyspin as a control for RNA loading.

FIG. 6 shows the dysregulation of the adipocyspin gene expression in obese mouse (ob/ob) 10 μg of total RNA from lean (ob/+) or obese (ob/ob) fat pads were analysed by Northern blot analysis. The same membrane was probed with ³²P labelled cDNAs corresponding to adipocyspin and adiponectin, as indicated. As in FIG. 5, the 18 S RNA hybridization signal is shown as a control for RNA loading.

FIG. 7 shows that adipocyspin inhibits the differentiation of 3T3 L1 preadipocytes. FLAG tagged adipocyspin was purified from transiently transfected COS 7 cells as in FIG. 4. 3T3 L1 cells were induced for differentiation in the absence of 20 μg/m FLAG-tagged adipocyspin (results are shown in A) or in the presence of 20 μg/ml FLAG-tagged adipocyspin (results are in B), where each slide is taken at day 6 after differentiation. Cells were stained with oil Red O and visualized by light microscopy.

FIG. 8 shows that adipocyspin inhibits the expression of adipocyte-specific gene products in adipocytes where 3T3 L1 cells were differentiated in the absence (lane 1) or presence (lane 2) of 20 μg/ml FLAG tagged adipocyspin. As in FIG. 7, the slides are taken 6 days after differentiation, where 10 μg of total RNA from these cells was analysed by Northern blotting as in FIG. 5, and probed with ³²P-labelled cDNAs corresponding to PPARγ or GLUT4.

FIG. 9 shows the cDNA sequences of mouse, rat, human and chicken adipocystins.

FIG. 10 shows the protein sequences of human and chicken adipocystins.

### DETAILED DESCRIPTION

The invention is not limited to the particular methods, protocols, cell lines, vectors, compositions and reagents described, as these may vary. It is also to be understood that the terminology used herein is for the purpose of describing particular embodiments only, and is not intended to limit the scope of the invention defined by the appended claims.

I. General Techniques

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, for example, Molecular Cloning: A Laboratory Manual, second edition (Sambrook et al., 1989) and Molecular Cloning: A Laboratory Manual, third edition (Sambrook and Russel, 2001), jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000).

II. Definitions

The terms “allele” or “allelic sequence,” as used herein, refer to a naturally-occurring alternative form of a gene encoding the adipocyspin polypeptide (i.e., a polynucleotide encoding an adipocyspin polypeptide). Alleles result from mutations (i.e., changes in the nucleic acid sequence), and sometimes produce altered and/or differently regulated mRNAs or polypeptides whose structure and/or function may or may not be altered. Common mutational changes that give rise to alleles are generally ascribed to natural deletions, additions, or substitutions of nucleotides that may or may not affect the encoded amino acids. Each of these types of changes may occur alone, in combination with the others, or one or more times within a given gene, chromosome or other cellular polynucleotide. Any given gene may have no, one or many allelic forms. As used herein, the term “allele” refers to either or both a gene or an mRNA transcribed from the gene.

As used herein, the term “amino acid” refers to naturally occurring and synthetic amino acids, as well as amino acid analogues, non-naturally occurring amino acids, and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogues refers to compounds that have the same basic chemical structure as a naturally occurring amino acid, i.e., an alpha-carbon that is bound to a hydrogen, a carboxyl group, an amino group, and an R group, e.g., homoserine, norleucine, methionine sulphoxide, methionine methyl sulphonium. Such analogues have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “antisense sequences” refers to polynucleotides having sequence complementary or desirably or usefully complementary to a RNA sequence. These terms specifically encompass nucleic acid sequences that bind to mRNA or portions thereof to block transcription of mRNA by ribosomes. Antisense methods are generally well known in the art (see, e.g., PCT publication WO 94/12633, and Nielsen et al., 1991, Science 254:1497; OLIGONUCLEOTIDES AND ANALOGUES, A PRACTICAL APPROACH, edited by F. Eckstein, IRL Press at Oxford University Press (1991); ANTISENSE RESEARCH AND APPLICATIONS (1993, CRC Press)).

The term “composition” as used herein is intended to encompass a product comprising the specified ingredients in specified or other amounts, as well as any product which results, directly or indirectly, from combination of the specified ingredients in specified or other amounts.

The term “conservative substitution,” when describing a polypeptide, refers to a change in the amino acid composition of the polypeptide that does not substantially alter the activity of the polypeptide, i.e., substitution of amino acids with other amino acids having similar properties such that the substitutions of even critical amino acids does not substantially alter activity. Conservative substitution tables providing functionally similar amino acids are well known in the art. The following six groups, for example, each contain amino acids that are understood to be conservative substitutions for one another: 1) Alanine (A), Serine (S), Threonine (1); 2) Aspartic acid (D), Glutamic acid (E); 3) Asparagine (N), Glutamine (Q); 4) Arginine (R), Lysine (K); 5) Isoleucine (1), Leucine (L), Methionine (N), Valine (V); and 6) Phenylalanine (F), T}Tyrosine (Y), Tryptophan (W) (see also, Creighton, 1984, Proteins, W.H. Freeman and Company).

In addition to the above-defined conservative substitutions, other modification of amino acid residues can result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, e.g. often less than 5%, in an encoded sequence. Further, a conservatively modified variant can be made from a recombinant polypeptide by substituting a codon for an amino acid employed by the native or wild-type gene with a different codon for the same amino acid.

The terms “control elements” or “regulatory sequences” include enhancers, promoters, transcription terminators, origins of replication, chromosomal integration sequences, 5′ and 3′ untranslated regions, with which polypeptides or other biomolecules interact to carry out transcription and translation. For eukaryotic cells, the control sequences will include a promoter and preferably an enhancer, e.g., derived from immunoglobulin genes, SV40, cytomegalovirus, and a polyadenylation sequence. and may include splice donor and acceptor sequences. Depending on the vector system and host utilized, any number of suitable transcription and translation elements, including constitutive and inducible promoters, may be used. When referring to an adipocyspin, a promoter other than that naturally associated with the adipocyspin coding sequence can be referred to as a “heterologous” promoter.

As used herein, a “derivatized” polynucleotide, oligonucleotide, or nucleic acid refers to oligo- and polynucleotides that comprise a derivatized substituent. In some embodiments, the substituent is substantially non-interfering with respect to hybridization to complementary polynucleotides. Derivatized oligo- or polynucleotides that have been modified with appended chemical substituents (e.g., by modification of an already synthesized oligo- or poly-nucleotide, or by incorporation of a modified base or backbone analogue during synthesis) may be introduced into a metabolically active eukaryotic cell to hybridize with an adipocyspin DNA, RNA, or protein where they produce an alteration or chemical modification to a local DNA, RNA, or protein. Alternatively, the derivatized oligo or polynucleotides may interact with and alter adipocyspin polypeptides, or proteins that interact with adipocyspin DNA or adipocyspin gene products, or alter or modulate expression or function of adipocyspin DNA, RNA or protein. Illustrative attached chemical substituents include, for example: europium (III) texaphyrin, cross-linking agents, psoralen, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, base-modification agents, immunoglobulin chains, and oligonucleotides. Iron/EDTA chelates are chemical substituents often used where local cleavage of a nucleic acid sequence is desired (Hertzberg et al., 1982. J. Am. Chem. Soc. 104: 313; Hertzberg and Dervan, 1984, Biochemistry 23: 3934; Taylor et al., 1984, Tetrahedron 40: 457; Dervan, 1986, Science 232: 464). Illustrative attachment chemistries include: direct linkage, e.g., via an appended reactive amino group (Corey and Schultz, 1988, Science 238: 1401) and other direct linkage chemistries, although streptavidin/biotin and digoxigenin/anti-digoxigenin antibody linkage methods can also be used. Methods for linking chemical substituents are provided in U.S. Pat. Nos. 5,135,720, 5,093,245, and 5,055,556. Other linkage chemistries may be used at the discretion of the practitioner.

As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use herein include, for example, any composition detectable by spectroscopic, photochemical, biochemical, immunochemical, electrical, optical, chemical means and the like.

The term “epitope” has its ordinary meaning of a site on an antigen recognized by an antibody. Epitopes are typically segments of amino acids which are a small portion of the whole polypeptide. Epitopes may be conformational (i.e., discontinuous). That is, they may be formed from amino acids encoded by noncontiguous parts of a primary sequence that have been juxtaposed by protein folding.

The term “fusion protein,” refers to a composite polypeptide, i.e., a single contiguous amino acid sequence, made up of two (or more) distinct, heterologous polypeptides which are not normally fused together in a single amino acid sequence. Thus, a fusion protein may include a single amino acid sequence that contains two entirely distinct amino acid sequences or two similar or identical polypeptide sequences, provided that these sequences are not normally found together in the same configuration in a single amino acid sequence found in nature. Fusion proteins may generally be prepared using either recombinant nucleic acid methods, i.e., as a result of transcription and translation of a recombinant gene fusion product, which fusion comprises a segment encoding a polypeptide and a segment encoding a heterologous polypeptide, or by chemical synthesis methods well known in the art.

The term “gene product” refers to an RNA molecule transcribed from a gene, or a polypeptide encoded by the gene or translated from the RNA.

The term “high affinity” for an IgG antibody, for example, as used herein, refers to an association constant (Ka) of at least about 10⁶ M⁻¹, preferably at least about 10⁸ M⁻¹, more preferably at least about 10⁹ M⁻¹ or greater, e.g., up to 10¹² M⁻¹ or greater. However, “high affinity” binding can vary for other antibody isotypes.

The terms “immunogen” and “immunogenic” have their ordinary meaning in the art, i.e., an immunogen is a molecule, such as a polypeptide or other antigen, that can elicit an adaptive immune response upon injection into a person or an animal.

The terms “modulator” and “modulation” of preadipocytes to adipocyte conversion activity, as used herein in its various forms, is intended to encompass antagonism, agonism, partial antagonism and/or partial agonism of the activity associated with a particular cell surface receptor, preferably the adipocyspin receptor. In various embodiments, “modulators” may inhibit or stimulate adipocyspin expression or activity. Such modulators include small molecules agonists and antagonists of adipocyspin function or expression; antisense and ribozyme triplex polynucleotides; gene therapy, and the like.

The terms “nucleic acid” and “polynucleotide” are used interchangeably and refer to deoxyribonucleotides or ribonucleotides and polymers thereof in either single- or double-stranded form. Unless specifically limited, the disclosure of a polynucleotide sequence is also intended to refer to the complementary sequence. As used herein, the term “polynucleotide” includes oligonucleotides.

The terms “oligonucleotides” or “oligomers” refer to a nucleic acid sequence of approximately 7 nucleotides or greater, and as many as approximately 100 nucleotides, for example, which can be used as, for example, a primer or probe. Oligonucleotides are often between about 10 and about 50 nucleotides in length) more often between about 12 and about 50 nucleotides, very often between about 15 and about 25 nucleotides.

The term “operably linked” refers to a functional relationship between two or more polynucleotide (e.g., DNA) segments: for example, a promoter or enhancer is operably linked to a coding sequence if it stimulates the transcription of the sequence in an appropriate host cell or other expression system. Generally, sequences that are operably linked are contiguous, and in the case of a signal sequence both contiguous and in reading phase. However, enhancers need not be located in close proximity to the coding sequences whose transcription they enhance.

The terms “peptidomimetic” and “mimetic” refer to a synthetic chemical compound that has substantially the same structural and functional characteristics of one or more adipocyspin polypeptides. Peptide analogues are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a adipocyspin, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2S0-. The mimetic can be either entirely composed of synthetic, non-natural analogues of amino acids, or, is a chimeric molecule of partly natural peptide amino acids and partly non-natural analogues of amino acids. The mimetic can also incorporate any amount of natural amino acid conservative substitutions as long as such substitutions also do not substantially alter the mimetic's structure and/or activity. For example, a mimetic composition is considered within the patent if it is capable of carrying out the binding or enzymatic activities of adipocyspin.

By “pharmaceutically acceptable” it is meant, for example, a carrier, diluent or excipient that is compatible with the other ingredients of the formulation and not deleterious or undesireably deleterious to the recipient thereof.

The term “polypeptide” is used interchangeably herein with the term “protein,” and refers to a polymer composed of amino acid residues linked by amide linkages, including synthetic, naturally-occurring and non-naturally occurring analogues thereof (amino acids and linkages). Peptides are examples of polypepfides.

As used herein, a “probe,” when used in the context of polynucleotides and antibodies, refers to a molecule that specifically or otherwise desirably binds another molecule. One example of a probe is a “nucleic acid probe,” which can be a DNA, RNA, or other polynucleotide. Where a specific sequence for a nucleic acid probe is given, it is understood that the complementary strand is also identified and included. The complementary strand will work equally well in situations where the target is a double-stranded nucleic acid that specifically binds (e.g., anneals or hybridizes) to a substantially complementary nucleic acid. Another example of a probe is an “antibody probe” that specifically binds to a corresponding antigen or epitope.

The term “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. Thus, a “recombinant” polynucleotide may be defined, for example, either by its method of production or its structure. In reference to its method of production, the process is use of recombinant nucleic acid techniques, e.g., involving human intervention in the nucleotide sequence, typically selection or production. Alternatively, it can be a polynucleotide made by generating a sequence comprising fusion of two fragments which are not naturally contiguous to each other, but is meant to exclude products of nature. Thus, for example, products made by transforming cells with any non-naturally occurring vector is encompassed, as are polynucleotides comprising sequence derived using any synthetic oligonucleotide process. Similarly, a “recombinant” polypeptide is one expressed from a recombinant polynucleotide.

The phrase “selectively hybridizing to” refers to a polynucleotide probe that hybridizes, duplexes or binds to a particular target DNA or RNA sequence to a desired degree when the target sequences are present in a preparation of total cellular DNA or RNA.

The phrase “specifically immunoreactive,” or “specifically binds” when referring to the interaction between an antibody and a protein or polypeptide, refers to an antibody that specifically recognizes and binds with relatively high affinity to the protein of interest, e.g., adipocyspin, such that this binding may be determinative of the presence of the protein in a heterogeneous population of proteins and other biologics. Thus, under designated immunoassay conditions, the specified antibodies bind to a particular polypeptide and do not bind in a significant or otherwise undesirable amount to other polypeptides present in the sample. A variety of immunoassay formats may be used to select antibodies that are immunoreactive or specifically immunoreactive with a particular polypeptide(s). For example, solid-phase ELISA immunoassays are routinely used to select monoclonal antibodies specifically immunoreactive with a polypeptide. See, Harlow, 1988, ANTIBODIES, A LABORATORY MANUAL, Cold Spring Harbor Publications, New York (hereinafter, “Harlow”), for a description of immunoassay formats and conditions that can be used to determine specific immunoreactivity.

As used herein, the “substantially sequence identity,” or “substantially identical” (in the context of comparing two or more polypeptides or polynucleotides) refers to two or more sequences or subsequences that have at least 60%, preferably 80%, most preferably 90%, 95%, 98%, or 99% nucleotide or amino acid residue identity, when compared and aligned for maximum correspondence, as measured using one of the following sequence comparison algorithms or by visual inspection. Two sequences (amino acid or nucleotide) can be compared over their full-length (e.g., the length of the shorter of the two, if they are of substantially different lengths) or over a subsequence such as at least about 50, about 100, about 200, about 500 or about 1000 contiguous nucleotides or at least about 10, about 20, about 30, about 50 or about 100 contiguous amino acid residues. Substantially identical polypeptides, as used herein, preferably have a common functional activity (e.g., biological activity).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. BioI. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols In Molecular Biology, Greene Publishing and Wiley-Interscience, New York (supplemented through 2001). When using any of the aforementioned algorithms, the default parameters for “Window” length. gap penalty, etc., are used.

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=4, and a comparison of both strands.

In addition to calculating percent sequence identity, the BLAST algorithm may also be used to perform a statistical analysis of the similarity between two sequences (see, e.g. Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the first polypeptide (e.g., a polypeptide encoded by the first nucleic acid) is immunologically cross reactive with the second polypeptide (e.g., a polypeptide encoded by the second nucleic acid). Thus, a polypeptide is typically substantially identical to a second polypeptide, for example, where the two peptides differ only by conservative substitutions.

Another indication that two nucleic acid sequences are substantially identical is that the two molecules hybridize to each other under stringent conditions. Substantial identity exists when the segments will hybridize under stringent hybridization conditions to a strand, or its complement, typically using a sequence of at least about 50 contiguous nucleotides derived from the probe nucleotide sequences.

A difference is typically considered to be “statistically significant” if the probability of the observed difference occurring by chance (the p-value) is less than some predetermined level. As used herein a “statistically significant difference” refers to a p-value that is <0.05. preferably <0.01 and most preferably <0.001.

“Stringent hybridization conditions” typically refers to conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, e.g., Berger and Kimmel, 1987, Methods In Enzymology, Vol. 152: Guide To Molecular Cloning Techniques) San Diego: Academic Press) Inc. and Sambrook et al.; supra; (1989) Molecular Cloning: A Laboratory Manual, 2nd Ed., Vols. 1-3, Cold Spring Harbor Laboratory). As indicated by standard references, a simple estimate of the Tm value may be calculated by the equation: Tm=81.5+0.41 (% G+C), when a nucleic acid is in aqueous solution at 1 M NaCI (see e.g., Anderson and Young, “Quantitative Filter Hybridization” in Nucleic Acid Hybridization (1985)). Other references include more sophisticated computations which take structural as well as sequence characteristics into account for the calculation of Tm. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art (see, e.g., Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30° C. for short probes (e.g., 10 to 50 nucleotides) and at least about 60° C. for long probes (e.g., greater than 50 nucleotides). As noted, stringent conditions may also be achieved with the addition of destabilizing agents such as formamide, in which case lower temperatures may be employed.

The terms “substantially pure” or “isolated,” when referring to proteins and polypeptides, e.g., an adipocyspin, denote those polypeptides that are separated in whole or in part from proteins or other contaminants with which they are naturally associated. A protein or polypeptide is considered substantially pure, for example, when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition. When referring to polynucleotides, the terms “substantially pure” or “isolated” generally refer to the polynucleotide separated from contaminants with which it is generally associated, e.g., lipids, proteins and other polynucleotides. Substantially pure or isolated polynucleotides will be greater than about 50% pure. Typically, these polynucleotides will be more than about 60% pure, more typically, from about 75% to about 90% pure and preferably from about 95% to about 98% pure.

The term “therapeutically effective amount” means the amount of the subject compound that will elicit the biological or medical response of a tissue, system, animal or human that is being sought by the researcher, veterinarian, medical doctor or other clinician.

As used herein, a “biological effect” includes the conversion of preadipocytes to adipocytes.

We have discovered and characterized a novel adipocyte-secreted factor, which we have named “adipocyspin” (with apparent molecular mass of 17 kDa and pI value of 9.4). It is expressed and secreted preferentially from 3T3 L1 adipocytes but not from corresponding preadipocytes. We also discovered following amino acid sequence analysis and cDNA cloning that this protein contains a putative secretory signal peptide, followed by a domain which shares some sequence homology with families of cysteine protease inhibitors. The adipocyspin mRNA was virtually undetectable in 3T3 L1 preadipocytes and markedly increased following hormone induced adipose conversion. Its expression in adipose tissue significantly increases in obese states.

The conversion of 3T3-L1 cells to adipocytes was dramatically inhibited following treatment with FLAG tagged adipocyspin purified from transiently transfected COS 7 cells. The regulated expression pattern and the inhibitory effect on adipocyte supports our discovery that adipocyspin has a role in the feedback regulation of adipogenesis. We further discovered that adipocyspin can down-regulate the conversion of preadipocytes into adipocytes. Uses for the compounds include, for example, administration or inhibition in states associated with decreased or increased or otherwise abnormal or undesired rates of conversion of preadipocytes to adipocyte conversion, such as those associated, for example, with adipocyte hyperplasia. Uses also include treatment of obesity and for weight reduction and/or prevention of weight gain.

Other uses include administration of an adiponectin or active fragment thereof. Administration may be, for example, in adiponectin-deficient states, or in states or conditions in which additional or increased adiponectin activity is desired.

In one aspect, polynucleotides are provided that have a sequence or subsequence of a mammalian (e.g., mouse, rat, or human) adipocyspin gene or DNA or RNA. The polynucleotides (e.g., RNA, DNA, PNA or chimeras) may be single-stranded, double stranded, or a mixed hybrid.

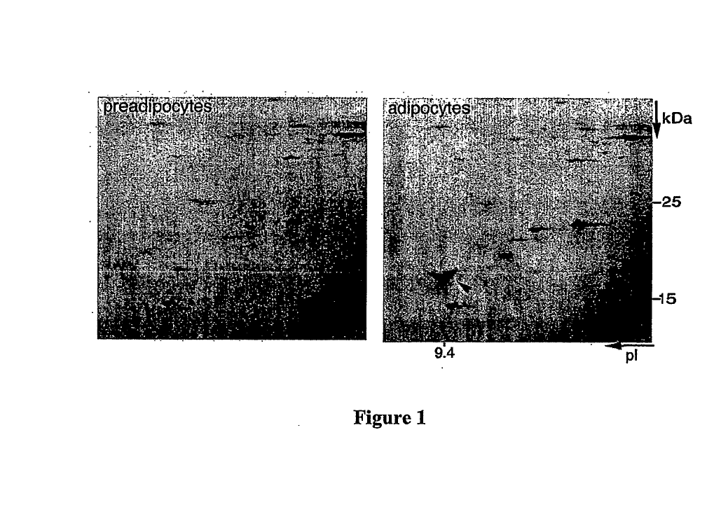

The polynucleotide may have a sequence that encodes a polypeptide of SEQ ID NO: 1 (FIG. 1) or SEQ ID NO: 2 or any subsequence thereof (e.g., comprising at least about 15, at least about 25, at least about 50, at least about 100, at least about 200, or at least about 500 bases of the polynucleotides and variants thereof). Polynucleotides with substantial sequence identity to the adipocyspin polynucleotides disclosed herein are also included. Thus, also provided, for example, are naturally occurring alleles of mammalian (e.g., human) adipocyspin genes such as human allelic variants of the adipocyspin polynucleotides encoding the molecule of SEQ ID NO:2.

As described herein, in some embodiments the polynucleotide encodes a polypeptide with substantial sequence similarity to SEQ ID NO:1 (FIG. 1), SEQ ID NO:2, SEQ ID NO:9, or SEQ ID NO:10 or encodes a bioactive or immunoactive fragment of such a polypeptide (including, e.g., a fusion protein), or a chimeric, mutated, and/or evolved form derived from such sequences. Also included are different polynucleotides that, due to the degeneracy of the genetic code, encode the polypeptide of SEQ ID NO:1 or SEQ ID NO:2 or a bioactive or immunoactive fragment thereof. In other embodiments, adipocyspin polynucleotides are provided that do not necessarily encode adipocyspin polypeptide but which are useful as, for example, probes, primers, antisense, triplex, RNAi or ribozyme reagents, and the like.

Also provided are expression vectors, cells, cell lines, and transgenic organisms comprising one or more adipocyspin polynucleotides encoding an adipocyspin and/or one or more bioactive or immunoactive fragments of such a polypeptide. In some embodiments, the vectors, cells, and organisms are capable of expressing the encoded adipocyspin polypeptides and fragments.

Adipocyspin polynucleotides and polypeptides, or fragments of either, can be produced by recombinant means. See, e.g., Sambrook et al., Berger and Kimmel, (1987) Methods In Enzymology, Vol. 152: Guide To Molecular Cloning Techniques, San Diego: Academic Press, Inc.; Ausubel et al., Current Protocols In Molecular Biology, Greene Publishing and Wiley-Interscience, New York (2001). Alternatively, adipocyspin polynucleotides, polypeptides, or fragments of either can be chemically synthesized using routine methods well known in the art (see, e.g., Narang et al., 1979, Meth. Enzymol. 68:90; Brown et al. 1979, Meth. Enzymol. 68:109; Beaucage et al., 1981, Tetra. Lett., 22:1859). In some embodiments, the adipocyspin polynucleotides contain, for example, non-naturally occurring bases, e.g., deoxyinosine (see, Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98) or modified backbone residues or linkages, e.g., peptide nucleic acids (PNA), methylphosphonate backbone, phosphorothioate backbone, and the like.

A polynucleotide encoding an adipocyspin may be subjected to any of various mutagenic or evolutionary methods to produce variants of the disclosed sequences. The variants may be selected to retain a biological activity of interest, and may be desirably altered in such activity. The selection process may occur after or during generation of such variants. Chemical or biological mutagenesis methods can be used, including chemical treatment of isolated nucleotides or of host cells, site-directed mutagenesis techniques, random biological mutagenesis methods including replication under conditions of poor fidelity, and directed evolution techniques. Polynucleotides can be subjected to DNA shuffling techniques such as those developed by Stemmer (U.S. Pat. Nos. 5,605,793; 5,811,238; 5,830,721) using sequences described herein. Polynucleotides can be subjected to systematic cassette mutagenesis methods such as those described by Huse. Systematic analysis of all mutants at each position of the protein can also be used, and can be done using a series of position-specific degenerate nucleotides (Short, U.S. Pats. Nos. 6,054,267, 5,939,250, 5,763,239, 6,537,776, 6,238,884, 6,171,820, 5,830,696 5,965,408, and 5,955,358). Biased mutagenesis methods can be used, including the use of libraries based on substitution matrices (U.S. Pat. Publ. 2020155460 A1 published Oct. 24, 2002 to Schellenberger et al.). Combinations of such methods are also provided. Methods of producing such mutants are provided, as are methods of use of adipocyspin described herein that incorporate such adipocyspin variants.

In one aspect, polynucleotides are provided that encode adipocyspin polypeptides and fragments thereof such as, for example, an adipocyspin polypeptide having the sequepce of SEQ ID NO:1 or SEQ ID NO:2, a bioactive or immunologically active (e.g., antigenic) fragment thereof, a variant thereof (e.g., a conservative or allelic variant), or an adipocyspin fusion polypeptide. In one embodiment, the polynucleotide comprises, consists essentially of, or consists of a polynucleotide encoding the sequence of SEQ ID NO:1, SEQ ID NO:2, SEQ ID NO:9, or SEQ ID NO:10 or a fragment thereof. In another aspect, the polynucleotides encode naturally occurring adipocyspin polypeptides or fragments that have differing sequences (e.g., as a result of species sequence distinctions and/or the degeneracy of the genetic code). In some embodiments, the polynucleotide is other than the expressed sequence tags H67224, AI1131555, AA215577, AW190975 or AI769466 or the polynucleotide encoding bovine PPR1 (Matsuoka et al., 1993, Biochem Biophys Res Comm 194:540-11).

Polynucleotides are useful, for example, for modulating expression of adipocyspin polynucleotides (e.g., sense or antisense DNAs and RNAs) and polypeptides, or fragments of either. Methods for recombinant expression of polynucleotides and polypeptides are well known in the art. Typically, the polynucleotides are used in expression vectors for the preparation of adipocyspin polypeptides and polynucleotides and fragments thereof. Expression vectors typically may include transcriptional and/or translational control signals (e.g., transcriptional regulatory element, promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells.

Thus, for example, DNA encoding an adipocyspin polypeptide or fragment may be inserted into DNA constructs capable of introduction into and expression in an in vitro host cell, such as a bacterial (e.g., E. coli, Bacillus subtilus), yeast (e.g., Saccharomyces), insect (e.g., Spodoprera frugiperda), or mammalian cell culture systems. Examples of mammalian cell culture systems useful for expression and production of the polypeptides include human embryonic kidney line (293; Graham et al., 1977, .I: Gen. Virol. 36:59); CHO (ATCC CCL 61 and CRL 9618); human cervical carcinoma cells (He La, ATCC CCL 2); and others known in the art. Useful human and nonhuman cell lines are widely available, e.g., from the American Type Culture Collection (ATCC), P.O. Box. 1549, Manassas, Va. 20108 (see http://www.atcc.org). The use of mammalian tissue cell culture to express polypeptides is discussed generally, for example, in Winnacker, FROM GENES TO CLONES (VCH Publishers, N.Y., N.Y., 1987) and Ausubel, supra.

In various systems, promoters from mammalian genes or from mammalian viruses may be used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV 40 promoter, and promoter-enhancer combinations known in the art.

Adipocyspin and polypeptides or fragments can also be expressed in transgenic animals (mouse, sheep, cow, etc.) and plants (tobacco, arabidopsis, etc.) using appropriate expression vectors that integrate into the host cell chromosome by art known methods.

Also provided are oligonucleotide or polynucleotide probes and/or primers for detecting or amplifying adipocyspin polynucleotides. The polynucleotides (e.g., probes and primers) may comprise or consist essentially of or consist of, for example, contiguous bases coding for at least about 5 amino acids within the polypeptide of SEQ ID NO:1 or SEQ ID NO:2, usually at least about 10 to 12 amino acids, typically at least about 15 amino acids, generally at least about 18 amino acids and often at least about 25, although they may also be 50 to 100 amino acids or more. When the adipocyspin polynucleotides are used as probes or primers they are generally less that about 3000 bases in length; typically they contain between about 12 and about 500 contiguous nucleotides that code for amino acids identical or exactly complementary to SEQ ID NO:1 or SEQ ID NO:2, more often between about 12 and about 50 contiguous nucleotides, even more often between about 15 and about 25 contiguous nucleotides.

The probes and primers may also be modified, e.g., by adding restriction sites to the probes or primers. The primers or probes may also comprise additional sequences, such as linkers. Additionally, primers or probes may be modified with detectable labels. For example, the primers and probes may be radiolabeled, may be chemically modified, e.g., derivatized, incorporate modified nucleotide bases, or may contain a ligand capable of being bound by an anti-ligand (e.g., biotin).

The adipocyspin probes and primers can be used for a number of purposes, e.g., for detecting or amplifying an adipocyspin polynucleotide in a biological sample, as discussed in more detail herein. For example, provided with the guidance herein, one of skill will be able to select primer pairs that specifically amplify all or a portion of the adipocyspin gene, mRNA, or cDNA in a sample. It is preferred that the primer pairs and amplification conditions are chosen to not amplify other polynucleotide sequences, such as other messenger RNAs present in the sample, e.g., due to complementarity between the 3′ end of the adipocyspin primers and other gene sequences.

Also provided are inhibitory polynucleotides such as antisense, triplex, and ribozyme and RNAi reagents that target or hybridize to adipocyspin polynucleotides.

In another aspect, antisense oligonucleotides and polynucleotides are provided that can be used to inhibit or down-regulate expression of an adipocyspin gene. Some therapeutic methods may involve the administration of an oligonucleotide that functions to inhibit and/or stimulate adipocyspin activity under in vivo physiological conditions, and is relatively stable under those conditions for a period of time sufficient for a therapeutic effect. Using art-known methods, polynucleotides can be modified to impart such stability and to facilitate targeting delivery of the oligonucleotide to the desired tissue, organ, or cell.

The antisense polynucleotides may comprise or consist essentially of an antisense sequence that specifically hybridizes to a sequence transcribed from the adipocyspin gene of at least about 10 bases, typically at least about 12 or 14, and up to about 800 contiguous nucleotides or the entire transcript length. More often, the antisense polynucleotide may be from about 12 to about 50 nucleotides in length or from about 15 to about 25 nucleotides in length. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate); among other factors.

Generally, to assure specific hybridization, the antisense sequence is substantially complementary to the target adipocyspin mRNA sequence. In certain embodiments, the antisense sequence is exactly complementary to at least a portion of the target sequence. The antisense polynucleotides may also include, however; nucleotide substitutions, additions, deletions, transitions, transpositions, or modifications, or other nucleic acid sequences or non-nucleic acid moieties so long as specific or otherwise desired binding to the relevant target sequence corresponding to adipocyspin RNA or its gene is retained as a functional property of the polynucleotide.

In one aspect, the antisense sequence is complementary to relatively accessible sequences of the adipocyspin mRNA (e.g.; relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner et al., 1997, Nature Biotechnology 15:537).

Also provided are antisense polynucleotides that have sequences in addition to the antisense sequence (i.e., in addition to an anti-adipocyspin-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, PNA modified, analogues, and the like) can be made using any suitable method for producing a nucleic acid, such as the chemical synthesis and recombinant methods disclosed herein. In one embodiment, for example, antisense RNA molecules may be prepared by de novo chemical synthesis or by cloning. For example, an antisense RNA that hybridizes to adipocyspin mRNA can be made by inserting (ligating) an adipocyspin DNA sequence (e.g., SEQ. ID NO:5 or 6, or a fragment thereof) in reverse orientation operably linked to a promoter in a vector (e.g., plasmid). Provided that the promoter and, preferably the termination and polyadenylation signals, are properly positioned, the strand of the inserted sequence corresponding to the noncoding strand will be transcribed and may act as an antisense oligonucleotide. The antisense oligonucleotides can be used to inhibit adipocyspin activity in cell-free extracts, cells, and animals, including mammals and humans.

For general methods relating to antisense polynucleotides, see ANTISENSE RNA AND DNA, (1988), D. A. Melton, Ed., Cold Spring Harbor Laboratory, Cold Spring Harbor, N.Y.) and Dagle et al., 1991, Nucleic Acids Research, 19:1805. For a review of antisense therapy, see, e.g., Uhlmann et al, Chem. Reviews, 90:543-584 (1990), and others.

Also provided are oligo- and polynucleotides (e.g., DNA, RNA, PNA, modified, analogues (or the like) that bind to double-stranded or duplex adipocyspin nucleic acids (e.g., in a folded region of the adipocyspin RNA or in the adipocyspin gene), forming a triple helix-containing, or “triplex” nucleic acid. Triple helix formation results in inhibition or down-regulation of adipocyspin expression by, for example, preventing transcription of the adipocyspin gene, thus reducing or eliminating adipocyspin activity in a cell. Without intending to be bound by any particular theory or mechanism of action, it is believed that triple helix pairing compromises the ability of the double helix to open sufficiently for the binding of polymerases, transcription factors, or regulatory molecules to occur.

Triplex oligo- and polynucleotides are constructed using the base-pairing rules of triple helix formation (see, e.g., Cheng et al., 1988. J: Biol. Chem. 263: 15110; FeIrin and Camerini-Otero. 1991, Science 354:1494; Ramdas et al., 1989, J: Biol. Chem. 264:17395; Strobel et al., 1991, Science 254:1639; and Rigas et al., 1986, Proc. Natl. Acad. Sci. U.S.A. 83: 9591) and the adipocyspin mRNA and/or gene sequence. Typically, the triplex-forming oligonucleotides comprise or consist essentially of or consist of a specific sequence of from about 10 to at least about 25 nucleotides or longer “complementary” to a specific sequence in the adipocyspin RNA or gene (i.e., large enough to form a stable triple helix, but small enough, depending on the mode of delivery, to administer in vivo, if desired). In this context, “complementary” means able to form a stable triple helix. In one embodiment, oligonucleotides bind specifically to the regulatory regions of the adipocyspin gene (e.g., the adipocyspin 5′-flanking sequence, promoters, and enhancers) or to the transcription initiation site, (e.g., between about −10 and about +10 from the transcription initiation site). For a review of recent therapeutic advances using triplex DNA, see Gee et al. in Huber and Carr, 1994, MOLECULAR. AND IMMUNOLOGIC APPROACHES, Futura Publishing Co, Mt Kisco N.Y. and Rininsland et al., 1997, Proc. Natl. Acad. Sci. USA 94:5854.

Also provided are ribozymes useful for inhibition or down regulation of adipocyspin activity. The ribozymes bind and specifically cleave and inactivate adipocyspin mRNA. Useful ribozymes can comprise 5′- and 3′-terminal sequences complementary to the adipocyspin mRNA and can be engineered by one of skill on the basis of the adipocyspin mRNA sequence disclosed herein (see PCT publication WO 93/23572, supra). Ribozymes include those having characteristics of group I intron ribozymes (Cech, 1995, Biotechnology 13:323), and others of hammerhead ribozymes (Edgington, 1992, Biotechnology 10:256).

Ribozymes include those having cleavage sites such as GUA, GUU, and GUC. Other optimum cleavage sites for ribozyme-mediated inhibition of adipocyspin activity in accordance include those described in PCT publications WO 94/02595 and WO 93/23569. Short RNA oligonucleotides between about 15 and about 20 ribonucleotides in length corresponding to the region of the target adipocyspin gene containing the cleavage site can be evaluated for secondary structural features that may render the oligonucleotide more desirable. The suitability of cleavage sites may also be evaluated by testing accessibility to hybridization with complementary oligonucleotides using ribonuclease protection assays, or by testing for in vitro ribozyme activity in accordance with standard procedures known in the art.

As described by Hu et al., PCT publication WO 94/03596, antisense and ribozyme functions can be combined in a single oligonucleotide. Moreover, ribozymes can comprise or consist essentially of or consist of one or more modified nucleotides or modified linkages between nucleotides, as described above in conjunction with the description of illustrative antisense oligonucleotides.

In one embodiment, ribozymes are generated in vitro and introduced into a cell or patient. In another embodiment, gene therapy methods are used for expression of ribozymes in a target cell ex vivo or in vivo.

Also provided are polynucleotides useful for inhibition of adipocyspin activity by methods such as RNA interference (RNAi), which may also include cosuppression and quelling. This and other techniques of gene suppression are well known in the art. A review of this technique is found in Science 288:1370-1372 (2000). RNAi operates on a post-transcriptional level and is sequence specific. The process comprises introduction of RNA with partial or fully double-stranded character, or precursors of or able to encode such RNA into the cell or into the extracellular environment.

As described by Fire et al., U.S. Pat. No. 6,506,559, the RNA may comprise one or more strands of polymerized ribonucleotide. The double-stranded structure may be formed by a single self-complementary RNA strand or two complementary RNA strands. The RNA may include modifications to either the phosphate-sugar backbone or the nucleosides for example, those described above in conjunction with the description of illustrative antisense oligonucleotides. RNA duplex formation may be initiated either inside or outside the cell.

Studies have demonstrated that one or more ribonucleases specifically bind to and cleave double-stranded RNA into short fragments. The ribonuclease(s) remain(s) associated with these fragments, which in turn specifically bind to complementary mRNA, i.e., specifically bind to the transcribed in RNA strand encoding for adipocyspin. The mRNA for the adipocyspin is also degraded by the ribonuclease(s) into short fragments, thereby obviating translation and expression of the adipocyspin gene, and so inhibiting or down-regulating adipocyspin activity. Additionally, an RNA polymerase may act to facilitate the synthesis of numerous copies of the short fragments, which exponentially increases the efficiency of the system. A unique feature of this gene suppression pathway is that silencing is not limited to the cells where it is initiated. The gene-silencing effects may be disseminated to other parts of an organism and even transmitted through the germ line to several generations.

Polynucleotides are provided which are useful for generating gene constructs for silencing adipocyspin genes. Polynucleotides may be used to generate genetic constructs that encode a single self-complementary RNA sequence specific for adipocyspin genes. Genetic constructs and/or adipocyspin-specific self-complementary RNA sequences may be delivered by any method known or discoverable in the art. Within genetic constructs, sense and antisense sequences flank an intron sequence arranged in proper splicing orientation making use of donor and acceptor splicing sites. Alternative methods may employ spacer sequences of various lengths rather than discrete intron sequences to create an operable and efficient construct. During post-transcriptional processing of the adipocyspin gene construct product, intron sequences are spliced-out, allowing sense and antisense sequences, as well as splice junction sequences, to bind forming double-stranded RNA. Select ribonucleases bind to and cleave the double-stranded RNA, thereby initiating the cascade of events leading to degradation of adipocyspin mRNA gene sequences, and silencing of adipocyspin gene(s).

Alternatively, rather than using a gene construct to express the self-complementary RNA sequences, the adipocyspin-specific double-stranded RNA segments are delivered to one or more targeted areas to be internalized into the cell cytoplasm to exert a gene silencing effect. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition or down-regulation. RNA containing a nucleotide sequences identical to a portion of the adipocyspin gene is preferred for inhibition or down-regulation. RNA sequences with insertions, deletions, and single point mutations relative to the target sequence have also been found to be effective for inhibition and down regulation. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

Therapeutic methods include the administration of an oligonucleotide that functions to inhibit, down-regulate, or stimulate adipocyspin activity under in vivo physiological conditions, and is relatively stable under those conditions for a period of time sufficient for a therapeutic effect. As noted above, modified nucleic acids may be useful in imparting such stability, as well as for targeting delivery of the oligonucleotide to the desired tissue, organ, or cell.

Oligo- and poly-nucleotides can be delivered directly as a drug in a suitable pharmaceutical formulation or indirectly by means of introducing a nucleic acid into a cell, including, for example, liposomes, immunoliposomes, ballistics, direct uptake into cells, and the like. For treatment of disease, the oligonucleotides will be administered to a patient in a therapeutically effective amount. A therapeutically effective amount is an amount sufficient to ameliorate the symptoms of the disease or modulate adipocyspin activity in the target cell. Methods useful for delivery of oligonucleotides for therapeutic purposes are described, for example, in U.S. Pat. No. 5,272,065. Other details of administration of pharmaceutically active compounds and compositions containing them are known or provided herein. In another embodiment, oligo- and poly-nucleotides can be delivered using gene therapy or other recombinant DNA expression strategies.

Gene therapy refers to the introduction of an otherwise exogenous polynucleotide which produces a medically useful phenotypic effect upon the (typically) mammalian cell(s) into which it is transferred. In one aspect, gene therapy methods and compositions are provided for treatment of adipocyspin-associated conditions. In illustrative embodiments, gene therapy involves, for example, introducing into a cell a vector that expresses or induces expression of an adipocyspin gene product (such as an adipocyspin protein substantially similar to the adipocyspin polypeptide having a sequence of SEQ ID NO:1 or 2, e.g., to increase adipocyspin activity, or an inhibitory adipocyspin polypeptide to reduce activity), expresses a nucleic acid having an adipocyspin gene or mRNA sequence (such as an antisense RNA, e.g., to reduce adipocyspin activity), expresses a polypeptide or polynucleotide that otherwise affects expression of adipocyspin gene products (e.g., a ribozyme directed to adipocyspin mRNA to reduce adipocyspin activity), or replaces or disrupts an endogenous adipocyspin sequence (e.g., gene replacement and gene knockout, insertion or deletion of regulatory sequence(s)). Numerous embodiments will be evident to one of skill upon review of the disclosure herein.

In one embodiment, a preparation comprising a physiologically acceptable carrier and a naked polynucleotide operatively coding for an adipocyspin polypeptide can be introduced to cause expression thereof. Compositions for introducing formulations comprising naked polynucleotides are described in PCT application No. WO 90/11092 (Vical Inc.), in PCT application No. WO 95/11307 (Institut Pasteur, INSERM, Universit d'Ottawa), and by Tacson et al. Nature Medicine. 2(8):888-892 (1996) and Huygen et al., Nature Medicine. 2(8):893-898 (1996). Bolistic methods can also be used.

Vectors useful in adipocyspin gene therapy can be viral or nonviral, and include those known, or described herein in relation to the adipocyspin expression systems. It will be understood by those of skill in the art that gene therapy vectors may comprise promoters and other regulatory or processing sequences, such as are described in this disclosure. Usually the vector will comprise a promoter and, optionally, an enhancer (separate from any contained within the promoter sequences) that serve to drive transcription of an oligo-ribonucleotide or polynucleotide, as well as other regulatory elements that provide for episomal maintenance or chromosomal integration and for high-level transcription, if desired. A plasmid useful for gene therapy can comprise other functional elements, such as selectable markers, identification regions, and other sequences. The additional sequences can have roles in conferring stability both outside and within a cell, targeting delivery of adipocyspin nucleotide sequences (sense or antisense) to a specified organ, tissue, or cell population, mediating entry into a cell, mediating entry into the nucleus of a cell and/or mediating integration within nuclear DNA. For example, aptamer-like DNA structures, or other protein binding moiety sites can be used to mediate binding of a vector to cell surface receptors or to serum proteins that bind to a receptor thereby increasing the efficiency of DNA transfer into the cell. Other DNA sites and structures can directly or indirectly bind to receptors in the nuclear membrane or to other proteins that go into the nucleus, thereby facilitating nuclear uptake of a vector. Other DNA sequences can directly or indirectly affect the efficiency of integration.

Suitable gene therapy vectors may, or may not, have one or more origins of replication. For example, it is useful to include an origin of replication in a vector for propagation of the vector prior to administration to a patient. However, the origin(s) of replication can be removed before administration if the vector is designed to integrate into the host chromosomal DNA or bind to host mRNA or DNA.

As noted, also provided are methods and reagents for gene replacement therapy (i.e., replacement by homologous recombination of an endogenous adipocyspin gene with a recombinant gene). Vectors specifically designed for integration by homologous recombination may be used. Factors for optimizing homologous recombination include, for example, the degree of sequence identity and length of homology to chromosomal sequences. The specific sequence mediating homologous recombination is also important because integration occurs much more easily in transcriptionally active DNA. Methods and materials for constructing homologous targeting constructs are described by, e.g., Mansour et al., 1988, Nature 336: 348; Bradley et al., 1992, Bio/Technology 10: 534. See also, U.S. Pat. Nos. 5,627,059; 5,487,992; 5,631,153; and 5,464,764. In one embodiment, gene replacement therapy may involve altering or replacing all or a portion of the regulatory sequences controlling expression of the gene that is to be regulated. For example, the adipocyspin promoter sequences (as showing in FIG. 5) may be disrupted to alter adipocyspin expression by inserting or deleting a transcriptional control site or inserting an exogenous promoter.

Also provided are methods and reagents for adipocyspin “gene knockout” in vitro and animals. This may be accomplished by deletion or disruption by homologous recombination of an endogenous adipocyspin gene using a recombinantly produced vector). In gene knockout, the targeted sequences can be regulatory sequences (e.g., the adipocyspin promoter), or RNA or protein coding sequences. The use of homologous recombination to alter expression of endogenous genes is described in detail, for example, in U.S. Pat. No. 5,272,071, WO 91/09955, WO 93/09222, WO 96/29411, WO 95/31560, and WO 91/12650. See also, Moynahan et al.; 1996, Hum. Mol. Genet. 5:875. Gene therapy vectors may be introduced into cells or tissues in vivo, in vitro, or ex vivo. For ex vivo therapy, vectors may be introduced into cells, e.g., stem cells, taken from the patient and clonally propagated for autologous transplant back into the same patient (see, e.g., U.S. Pat. Nos. 5,399,493 and 5,437,994).

Also provided are transgenic non-human multicellular organisms (e.g., plants and non-human animals) or unicellular organisms (e.g., yeast) comprising an exogenous adipocyspin gene sequence, which may be a coding sequence or a regulatory (e.g., promoter) sequence. Examples of multicellular organisms include plants, insects, and nonhuman animals such as mice, rats, rabbits, monkeys, apes, pigs, and other nonhuman mammals. An example of a unicellular organism is a yeast. In one embodiment, the organism expresses an exogenous adipocyspin polypeptide, having a sequence of a human adipocyspin protein. Also provided are unicellular and multicellular organisms (or cells therefrom) in which a gene encoding adipocyspin is mutated or deleted (e.g., in a coding or regulatory region) such that native adipocyspin is not expressed, or is expressed at reduced levels or with different activities when compared to wild-type cells or organisms. Such cells and organisms are often referred to as “gene knock-out” cells or organisms.

The patent further provides cells and organisms in which an exogenous adipocyspin gene or variant (e.g., human adipocyspin) is introduced and expressed. The exogenous adipocyspin gene or variant may augment or replace an endogenous adipocyspin gene. Cells and organisms of this type can be used, for example, as model systems for identifying modulators of adipocyspin activity or expression or determining the effects of mutations in the adipocyspin gene.

Methods for alteration or disruption of specific genes (e.g., endogenous adipocyspin genes) are well known to those of skill, see, e.g., Baudin et al., 1993. Nucl. Acids Res. 21:3329; Wach et al., 1994, Yeast 10:1793; Rothstein, 1991, Methods Enzymol. 194:281; Anderson, 1995, Methods Cell Biol. 48:31; Pettitt et al. 1996, Development 122:4149-4157; Ramirez-Solis et al., 1993, Methods Enzymol. 225:855; and Thomas et al., 1987, Cell 51:503. Typically, such methods involve, for example, altering or replacing all or a portion of the regulatory sequences controlling expression of the particular gene to be regulated. The regulatory sequences, e.g. the native promoter, can be altered. One conventional technique for targeted mutation of genes involves placing a genomic DNA fragment containing the gene of interest into a vector, followed by cloning of the two genomic arms associated with the targeted gene around a selectable neomycin-resistance cassette in a vector containing thymidine kinase. This “knock-out” construct is then transfected into the appropriate host cell, i.e., a mouse embryonic stem (ES) cell, which is subsequently subjected to positive selection (using G418, for example, to select for neomycin-resistance) and negative selection (using, for example, FIAU to exclude cells lacking thymidine kinase), allowing the selection of cells which have undergone homologous recombination with the knockout vector. This approach leads to inactivation of the gene of interest. See, for example, U.S. Pat. Nos. 5,464,764; 5,631,153; 5,487,992; and, 5,627,059.

The “knocking out” expression of an endogenous gene can also be accomplished by the use of homologous recombination to introduce a heterologous nucleic acid into the regulatory sequences (e.g., promoter) of the gene of interest. To prevent expression of the functional enzyme or product, simple mutations that either alter the reading frame or disrupt the promoter can be suitable. Also, “gene trap insertion” can be used to disrupt a host gene, and mouse ES cells can be used to produce knockout transgenic animals, as described for example, in Holzschu (1997) Transgenic Res 6: 97-106. Other methods are known in the art.

To up-regulate expression, a native promoter can be substituted with a heterologous promoter that induces higher levels of transcription.

Altering the expression of endogenous genes by homologous recombination can also be accomplished by using nucleic acid sequences comprising the structural gene in question. Upstream sequences are utilized for targeting heterologous recombination constructs. Utilizing adipocyspin structural gene sequence information, such as the genomic polynucleotide sequence that encodes the polypeptide of SEQ ID NO:1 or SEQ ID NO:2, one of skill in the art can create homologous recombination constructs with only routine experimentation.

Homologous recombination to alter expression of endogenous genes is described, for example, in U.S. Pat. No. 5,272,071, and WO 91/09955, WO 93/09222, WO 96/29411, WO 95/31560, and WO 91/12650. Homologous recombination in animals has been described by Moynahan (1996) Hum. Mol. Genet. 5:875, and in plants by Offringa (1990) EMBO J. 9:3077.

Also provided are isolated, substantially pure, or recombinant adipocyspin polypeptides and immunogenic fragments of adipocyspin polypeptides. In one embodiment, the adipocyspin polypeptide or fragment has an amino acid sequence identical to, or substantially identical to, the sequence set forth in SEQ ID NO:1 or SEQ ID NO:2 or a subsequence thereof.

Also provided are substantially pure, isolated, or recombinant adipocyspin polypeptides. In some embodiments, the adipocyspin polypeptide has an amino acid sequence identical or substantially identical to the amino acid sequence shown in SEQ ID NO:1 or SEQ ID NO:2. In other embodiments, the adipocyspin polypeptides are variants and mutants characterized by conservative substitutions of amino acid residues of SEQ ID NO:1 or SEQ ID NO:2.

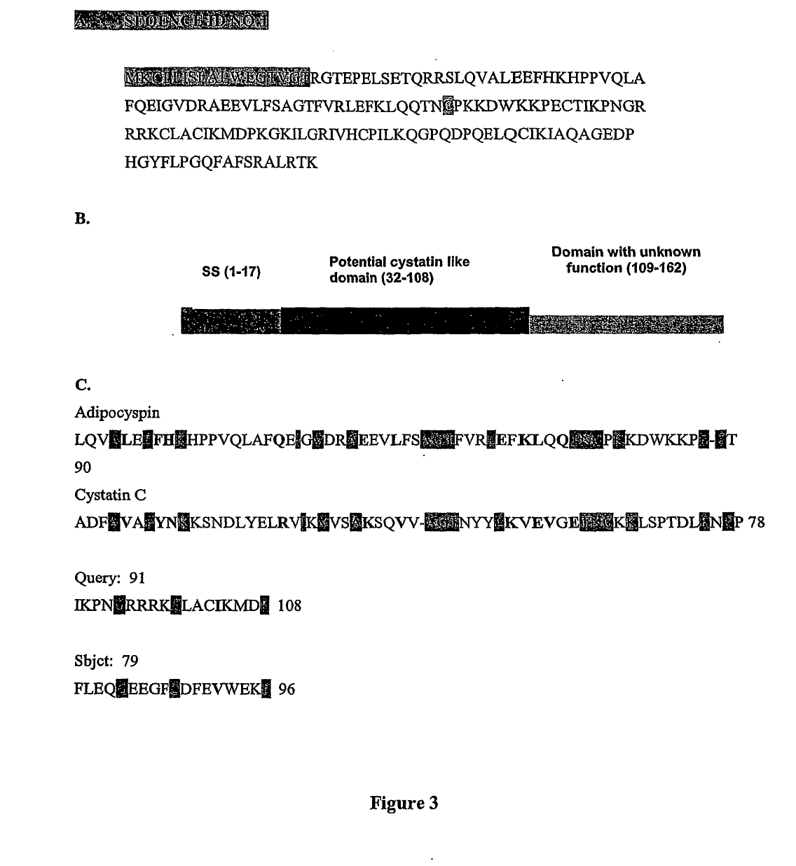

The polypeptide may be full-length (e.g. containing about 150 amino acids for the species shown in FIG. 3 or may encode a fragment of the full-length protein (e.g., comprising at least 20, at least 40, at least 60 or at least 100 residues of the adipocyspin polypeptides and variants. Also provided are adipocyspin polypeptides that are modified, relative to the amino acid sequence of SEQ ID NO:1, SEQ ID NO:2, SEQ ID NO:9 or SEQ ID NO:10, in some manner; e.g., truncated, mutated, derivatized; or fused to other sequences (e.g.; to form a fusion protein). Some adipocyspin polypeptides comprise insertions, deletions or substitutions of amino acid residues relative to the sequences disclosed. For example, conservative amino acid substitutions can be made, i.e., substitution of selected amino acids with different amino acids having similar structural characteristics, e.g., net charge, hydrophobicity, polarity, size, and the like.

The adipocyspin variants may be structurally and functionally similar to the adipocyspin polypeptide of SEQ ID NO:1, SEQ ID NO: 2, SEQ ID NO:9 or SEQ ID NO:10. Structural similarity is indicated by, e.g., substantial sequence identity (as defined above), or immunological cross-reactivity. Functional similarity is indicated by, e.g., inhibition of conversion of preadipocytes into adipocytes.

In some embodiments, adipocyspin variants may comprise at equivalent amino acid positions at least two of the cysteine residues at amino acid positions 62, 72, 83, 86, 101 and 116 of human adipocyspin as shown in FIG. 3B. One cysteine residue may be joined to form an intramolecular disulphide bond with another cysteine residue, such that adipocyspin and/or adipocyspin isoforms may contain up to 3 intramolecular disulphide bonds. Adipocyspin variants that are modified relative to the amino acid sequence of SEQ ID NO:1 or SEQ ID NO:2 as described above may be capable of forming other intramolecular or intermolecular disulphide bonds, for example, they may comprise other cysteine residues capable of forming intramolecular disulphide bonds. Adipocyspin variants may also be modified to eliminate one or more disulphide bonds, for example by mutating or deleting one or more cysteine residues, which may improve its stability, oxygen sensitivity, or pharmacological properties.

In some embodiments, adipocyspin polypeptides may be transcriptionally, post-transcriptionally or post-translationally modified yielding various adipocyspin variants and/or isoforms. Such modifications are well known in the art, and can include for example alternative splicing, RNA editing, proteolytic cleavage, glycosylation, phosphorylation, pegylation, acylation, methylation, sulfation, prenylation, and the like. The polypeptides may have sites introduced via genetic and/or chemical methods that can be glycosylated or pegylated, which may improve the biological half-life or other pharmacological properties of adipocyspin.

In some embodiments, an adipocyspin polypeptide or fragment thereof may be used as an immunogen (e.g., to produce anti-adipocyspin antibodies). Typically, the immunogenic adipocyspin fragments comprise at least about 6 or more contiguous residues of SEQ ID NO:1, SEQ ID NO:2, SEQ ID NO:9 or SEQ ID NO:10, more often at least about 8, about 10, or about 12, or about 16 contiguous residues, for example.

Substantially pure, isolated or recombinant adipocyspin polypeptides can also be characterized by their ability to bind antibodies that are specifically immunoreactive with a polypeptide having the sequence shown in SEQ ID NO:1 or SEQ ID NO:2. Specific immunoreactivity is usually characterized by a specific binding affinity of an antibody for its ligand (e.g., adipocyspin) of at least about 10⁷, 10⁸, 10⁹; or 10¹⁰ M⁻¹, for example.

For many applications, it will also be desirable to provide adipocyspin polypeptides as labelled entities, i.e., covalently attached or linked to a detectable label or group, or cross-linkable group, to facilitate identification, detection or quantification of the polypeptide in a given circumstance. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group, for example. Similarly, the detectable group can be a substrate, cofactor, inhibitor or affinity ligand.

In addition, an adipocyspin polypeptide can be modified by substituting one or more amino acid residues with a D-amino acid of the same type (e.g., D-lysine in place of L-lysine) to generate, for example, peptides with additional stability. Similarly, modification of the amino or carboxyl terminals can also be used to confer stabilizing properties upon the polypeptides, e.g., amidation of the carboxyl-terminus or acylation of the amino-terminus or pegylated derivatives.

Adipocyspin polypeptides can be prepared using recombinant or synthetic methods, or can be isolated from natural cellular sources, for example.

Suitable recombinant techniques for expressing adipocyspin polypeptides from the adipocyspin polynucleotides are known, or disclosed herein. See also, Sambrook et al., 1989, MOLECULAR CLONING: A LABORATORY MANUAL, (2nd ed.) Vols. 1-3, Cold Spring Harbor Laboratory, and in Ausubel, supra. Synthetic methods for synthesizing polypeptides such as adipocyspin polypeptides, variants, or fragments are described, for example, in Merrifield˜1963) Amer. Chem. Soc. 85:2149-2456, Atherton et al., 1989, SOLID PHASE PEPTIDE SYNTHESIS: A PRACTICAL APPROACH, IRL Press, and Merrifield, 1986, Science 232:341-347.

Isolation and purification of the adipocyspin polypeptides can be carried out by methods that are generally well known in the art. These methods include, but are not limited to, ion exchange, hydrophobic interaction, HPLC, or affinity chromatography, to achieve the desired purity. In one embodiment, adipocyspin polypeptides may be purified using immunoaffinity chromatography. For example, antibodies raised against a adipocyspin polypeptide or immunogenic fragment thereof (e.g., having a sequence or subsequence of SEQ ID NO:1 or SEQ ID NO:2) are coupled to a suitable solid support and contacted with a mixture of polypeptides containing the adipocyspin polypeptide (e.g., a homogenate of adipose tissue) under conditions conducive to the association of this polypeptide with the antibody. Once the adipocyspin polypeptide is bound to the immobilized antibody, the solid support is washed to remove unbound material and/or nonspecifically bound polypeptides. The desired polypeptide can then be eluted from the solid support in substantially pure form by, e.g., a change in pH or salt concentration of the buffer.

Although primarily described in terms of “proteins” or “polypeptides,” one of skill in the art will understand that structural analogues and derivatives of the above-described polypeptides, e.g., peptidomimetics, and the like can be used as substitutes for adipocyspin, e.g., as adipocyspin agonists, or, alternatively, as adipocyspin antagonists. Peptidomimetics, or peptide mimetics, are peptide analogues commonly used in the pharmaceutical industry as non-peptide drugs with properties (e.g., a biological activity) analogous to those of the template peptide (Fauchere, 1986, Adv. Drug Res. 15:29; Evans et al., 1987, J. Med. Chem. 30:1229). They are usually developed, for example, with the aid of computerized molecular modelling. Peptide mimetics that are structurally similar to therapeutically useful peptides can be used to produce an equivalent or substantially equivalent therapeutic effect. Peptide mimetics can have significant advantages over polypeptide embodiments, including, for example, more economical production and greater chemical stability.

Also provided are antibodies or antibody binding fragments, including scFvs, that are specifically or otherwise desirably immunoreactive with a mammalian adipocyspin polypeptide, for example, a rodent or a human adipocyspin. Accordingly, the antibodies or binding fragments may specifically recognize and bind polypeptides which have an amino acid sequence identical, or substantially identical, to the amino acid sequence of SEQ ID NO:1 or SEQ ID NO:2, or an immunogenic fragment thereof. The antibodies usually exhibit a specific binding affinity for adipocyspin of at least about 10⁷, 10⁸, 10⁹, or 10¹⁰ M⁻¹, for example.

The anti-adipocyspin antibodies and fragments have a variety of uses, e.g., isolation of adipocyspin polypeptides (e.g., by immunoaffinity chromatography), detection of adipocyspin polypeptides, and for inhibition of adipocyspin activity (e.g., in vivo or in vitro).

Anti-adipocyspin antibodies can be made by a variety of means well known to those of skill in the art, e.g., as described supra. As noted, antibodies are broadly defined herein and include fragments, chimeras and similar binding agents (e.g., the products of phage display technology), that specifically, or otherwise desirable, binds an adipocyspin polypeptide or epitope. In one embodiment, the antibody includes recombinantly-prepared single or double-chain or multiple-chain polypeptides containing antibody light and heavy chain variable domains sufficient for antigen-specific binding, and at least a fragment of antibody light and heavy chain constant regions (e.g., the C_{H}¹ domain of the heavy chain) sufficient in the case of a double-chain polypeptide to maintain association of the two polypeptides. In one embodiment, the antibody is a single chain antibody (sFv), for example comprising antibody light and heavy chain variable domains, typically joined by a suitable linker configured to bind adipocyspin or an adipocyspin eptitope.

Methods for production of polyclonal or monoclonal antibodies are well known in the art. See, e.g., Coligan, CURRENT PROTOCOLS IN IMMUNOLOGY, Wiley/Greene, N.Y. (1991); Stites et al. (eds.) BASIC AND CLINICAL IMMUNOLOGY (7th ed.) Lange Medical Publications, Los Altos, Calif., and references cited therein (“Stites”); Goding, MONOCLONAL ANTIBODIES: PRINCIPLES AND PRACTICE (2d ed.) Academic Press, New York, N.Y. (1986); Kohler and Milstein, 1975t Nature 256:495-97; and Harlow and Lane. These techniques include antibody preparation by selection of antibodies from libraries of recombinant antibodies in phage or similar vectors. See, Huse et al., 1989, Science 246:1275-81; and Ward et al., 1989, Nature 341:544-46.

For production of polyclonal antibodies, an appropriate target immune system is selected, typically a mouse or rabbit, but also including goats, sheep, cows, chickens, guinea pigs, monkeys and rats. The immunoglobulins produced by the host can be precipitated, isolated and purified by routine methods, including affinity purification. Substantially monospecific antibody populations can be produced by chromatographic purification of polyclonal sera, if desired.

For monoclonal antibodies appropriate animals will be selected and the desired immunization protocol followed. The antibodies may be of any isotype, e.g., IgM, IgD, IgG IgA, and IgE, with IgG, IgA and IgM being preferred and IgG most referred. Preferred monoclonal anti-adipocyspin antibodies neutralize (i.e., inhibit or block) one or more biological activities of adipocyspin. Such antibodies may be obtained by screening hybridoma supernatants for the desired 20 inhibitory activity. Monoclonal antibodies with affinities of 10⁸ liters/mole, preferably 10⁹ to 10¹⁰ or stronger, can be produced by the methods described below. The production of non-human monoclonal antibodies, e.g., murine, lagomorpha, or equine, is well known and can be accomplished by, e.g., immunizing a host animal with a preparation containing adipocyspin or fragments thereof. Antibody-producing cells obtained from the immunized animals are immortalized and screened, or screened first for the production of antibody which binds to the adipocyspin polypeptide and then immortalized.

Monoclonal antibodies may be humanized or made chimeric using techniques known in the art. Some anti-adipocyspin monoclonal antibodies are humanized, human, or chimeric, in order to reduce their potential antigenicity, without reducing their affinity for their target. Humanized antibodies may be prepared as described in the art. See, e.g. 30 Queen, et al., 1989, Proc. Nat'l Acad. Sci. USA 86:10029; U.S. Pat. Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., Protein Engineering 4:773 (1991); Kolbinger et al., Protein Engineering 6:971 (1993). Humanized monoclonal antibodies against adipocyspin can also be produced using transgenic animals having elements of a human immune system (see, e.g., U.S. Pat. Nos. 5,569,825; 5,545,806; 5,693,762; 5,693,761; and 5,7124,350).

Useful anti-adipocyspin antibodies can also be produced using phage display technology (see, e.g., Dower et al., WO 91/17271 and McCafferty et al., WO 92/01047). In these methods, libraries of phage are produced in which members display different antibodies on their outer surfaces. Antibodies are usually displayed as Fv or Fab fragments. Phage displaying antibodies with a desired specificity are selected by affinity enrichment to an adipocyspin polypeptide. Single chain antibodies can be produced using methods well known in the art (see, e.g., Colcher et al. (1999) Ann. N Y Acad. Sci. 880:263-80; Reiter (1996) Clin. Cancer Res. 2:245-52); U.S. Pat. Nos. 4,946,778; 5,260,203; 5,455,030; 5,518,889; and 5,534,621).

Once expressed, the whole antibodies, their dimers, individual light and heavy chains, or other immunoglobulin forms can be purified according to standard procedures of the art, including ammonium sulphate precipitation, affinity chromatography, gel electrophoresis and the like (see generally PROTEIN PURIFICATION: PRINCIPLES AND PRACTICE 3RD EDITION (Springer-Verlag, N.Y., 1994)).

An antibody (e.g. an anti-adipocyspin antibody or any fragment thereof), may be substantially pure when at least about 80%, more often at least about 90%, even more often at least about 95%, most often at least about 99% or more of the polypeptide molecules present in a preparation specifically bind the Same antigen (e.g. adipocyspin polypeptide). For pharmaceutical uses, anti-adipocyspin immunoglobulins of at least about 90 to 95% homogeneity are preferred, and 98 to 99% or more homogeneity are most preferred.

The antibodies can be used with or without modification. Frequently, the antibodies will be labelled by joining, either covalently or non-covalently, a substance which provides for a detectable signal. Such labels include those that are well known in the art, e.g., radioactive, fluorescent, or bioactive (e.g., enzymatic) labels. As labelled binding entities, the antibodies may be particularly useful in diagnostic applications.

Also provided are hybrid or “bispecific” antibodies that share the specificity of antibodies against an adipocyspin polypeptide but are also capable of specific binding to a second moiety. In hybrid antibodies, one heavy and light chain pair is from one antibody and the other pair from an antibody raised against another epitope. This results in the property of multi-functional valency, i.e., ability to bind at least two different epitopes simultaneously. Such hybrids can be formed by fusion of hybridomas producing the respective component antibodies, or by recombinant techniques.

In some embodiments, an anti-adipocyspin monoclonal or polyclonal antiserum may be produced that is specifically or desirably immunoreactive with adipocyspin and is selected to have low or a desired level of crossreactivity against other proteins, for example, homologous proteins such as cystatin; any such crossreactivity can be removed from a polyclonal antiserum by immunoabsorption prior to use in the immunoassay. Methods for screening and characterizing monoclonal antibodies for specificity are well known in the art and are described generally in Harlow and Lane, supra. In order to produce a polyclonal antisera (e.g., for use in an immunoassay), against the protein of SEQ ID NO:1 or SEQ ID NO:2, for example, a polyclonal antiserum is prepared using methods well known in the art, including methods such as those described herein. For example, recombinant protein may be produced in a mammalian cell line. An inbred strain of mice such as balb/c is immunized with the protein of SEQ ID NO:1 or SEQ ID NO:2 using a standard adjuvant, such as Freund's adjuvant, and a standard mouse immunization protocol (see Harlow and Lane, supra). Alternatively, a synthetic peptide derived from one or more of the sequences disclosed herein and conjugated to a carrier protein can be used as an immunogen. Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against other human cysteine proteases (e.g., one or more of cystatin or known homologues) using a competitive binding immunoassay such as the one described in Harlow and Lane, supra, at pages 570-573. Immunoassays in the competitive binding format can be used for the crossreactivity determinations. For example, adipocyspin can be immobilized to a solid support. Proteins added to the assay compete with the binding of the antisera to the immobilized antigen. The ability of the above proteins to compete with the binding of the antisera to the immobilized protein is compared to adipocyspin. The percent cross reactivity for the above proteins is calculated using any suitable method. Those antisera with less than 10% crossreactivity with each of the proteins listed above are selected and pooled. The cross-reacting antibodies are then removed from the pooled antisera by immunoabsorption with the above-listed proteins.

In one aspect, expression of adipocyspin may be monitored or determined for diagnosis or evaluation of an individual with a disease state or a propensity toward a disease state associated with adipocyspin regulation. In various embodiments, the disease state is obesity and/or diabetes, for example, type 2 diabetes, and/or the condition known as “Syndrome X” or “Metabolic Syndrome”, including aberrant glucose sensitivity and/or glucose insensitivity.

The adipocyspin can be obtained from a biological fluid, e.g., serum, plasma, urine, saliva or blood and analyzed, for example, by electrophoresis, HLPC, mass spectrometry, immunologically, etc. The expression profile can be monitored by any of the methods disclosed herein or known in the art.

The monitoring can be accomplished by monitoring the level of adipocyspin in an individual. In one embodiment, the level or expression profiles in an individual are compared with a control, where a statistically significant correlation with the control is diagnostic of a condition.

Also provided are assay methods involving screening molecules for the ability to modulate the activity of the adipocyspin. Included are methods to evaluate putative specific agonists or antagonists of adipocyspin function. Of particular interest are molecules that modulate the conversion of preadipocytes into adipocytes. Accordingly, also contemplated is the use of these compounds in the preparation and execution of screening assays for compounds which modulate the ability of adipocyspin to prevent the conversion of preadipocytes into adipocytes. Exemplary molecules which may be assayed using such methods include organic molecules, inorganic molecules, polymers, small molecules, polynucleotides including antisense and siRNA molecules or precursors thereto, variants and altered forms of adipocystin, and antibodies.

Preliminary screens can be conducted by screening for compounds capable of binding to an adipocyspin receptor or binding partner, as at least some of the compounds so identified are likely adipocyspin modulators. The binding assays usually involve, for example, contacting an adipocyspin agonist such as adipocyspin protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The adipocyspin protein utilized in such assays can be naturally expressed, cloned or synthesized adipocyspin. In one embodiment, the assay is a cell-based assay and cells are used which are stably or transiently transfected with a vector or expression cassette having a nucleic acid sequence which encodes an adipocyspin receptor or binding partner. The cells are maintained under conditions appropriate for expression of the adipocyspin receptor and are contacted with a putative agent under conditions appropriate for binding to occur. Binding can be detected using standard techniques. For example, the extent of binding can be determined relative to a suitable control (for example, relative to background in the absence of a putative agent, or relative to a known ligand). Optionally, a cellular fraction, such as a membrane fraction, containing the receptor can be used in lieu of whole cells.

Detection of binding or complex formation can be detected directly or indirectly. For example, the putative agent can be labelled with a suitable label (e.g., fluorescent label, chemiluminescent label, isotope label, enzyme label, and the like) and binding can be determined by detection of the label. Specific and/or competitive binding can be assessed by competition or displacement studies, using unlabelled agent as a ligand (e.g., recombinant adipocyspin) as a competitor.

In other embodiments, binding inhibition assays can be used to evaluate compounds. In these assays, the compounds are evaluated as inhibitors of ligand binding using, for example, adipocyspin receptor expressed in NIH 3T3 L1 cells. In this embodiment, the adipocyspin receptor is contacted, for example, with a ligand such as adipocyspin, and a measure of ligand binding is made. The receptor is then contacted with a test agent in the presence of a ligand (e.g., recombinant adipocyspin) and a second measurement of binding is made. A reduction in the extent of ligand binding is indicative of inhibition of binding by the test agent. Binding inhibition assays can be carried out using whole cells which express the adipocyspin receptor, or a membrane fraction from cells which express adipocyspin receptor.

Certain screening methods involve screening for a compound that up-regulates (or, alternatively, inhibits or down-regulates) the expression or activity of adipocyspin. Such methods generally involve conducting cell-based assays in which test compounds are contacted with one or more cells expressing adipocyspin and then detecting a change (e.g., increase or decrease) in adipocyspin expression (either transcript or translation product) or activity. Some assays are performed, for example, with cells that express endogenous adipocyspin (e.g., adipocytes or NIH 3T3 L1 cells). Other expression assays may be conducted with recombinant cells that express adipocyspin encoded in a suitable expression vector. In either case, adipocyspin expression can be detected in a number of different ways, as described herein. For example, the expression level of an adipocyspin in a cell can be determined by probing the mRNA expressed in a cell with a probe that specifically hybridizes with a transcript (or complementary nucleic acid derived therefrom) encoding an adipocyspin. Probing can be conducted by lysing the cells and conducting Northern blots or without lysing the cells using in situ-hybridization techniques (see above). Alternatively, adipocyspin protein can be detected using immunological methods in which a cell lysate is probed with antibodies that specifically bind to adipocyspin. Similarly, adipocyspin activity can be assayed by determining, for example, the rate of conversion of adipocyte precursors into adipocytes, for example pre-NIH 3T3 L1 cells into NIH 3T3 L1 adipocytes. One method of drug screening utilizes eukaryotic or prokaryotic host cells which are stably transformed with recombinant DNA molecules expressing adipocyspin, e.g. the protein having the sequence of SEQ ID NO:1 or SEQ ID NO:2. Such cells, either in viable or fixed form, can be used for screening. A test compound can be assayed for binding or for competition with another ligand for binding.

In one example of a suitable assay, an adipocyspin protein (whether isolated or recombinant) is used which has at least one property, activity or functional characteristic of a human adipocyspin protein. The property can be, for example, suppression of conversion of preadipocytes into adipocytes.

In one embodiment, a composition containing an adipocyspin protein or variant thereof is maintained under conditions suitable for binding. The adipocyspin receptor is contacted with a putative agent (or a second composition containing at least one putative agent) to be tested and binding is detected or measured.

The level of expression or activity can be compared to a baseline value. Expression levels can also be determined for cells that do not express adipocyspin as a negative control. Such cells generally are otherwise substantially genetically the same as the test cells.

The test compounds, adipocyspin activity modulators or putative modulators and other compounds provided herein can also be evaluated using models of inflammation to assess the ability of the compound to exert an effect in vivo. Suitable models include, for example, the following:

Several methods of automating assays have been developed in recent years so as to permit screening of tens of thousands of compounds in a short period. See, e.g., Fodor et al., 1991, Science 251: 767-73, and other descriptions of chemical diversity libraries, which describe means for testing of binding affinity by a plurality of compounds. The development of suitable assays can be greatly facilitated by the availability of large amounts of purified adipocyspin and/or cells expressing recombinant adipocyspin as provided by this invention.

The terms “upregulated” and “activation” when used in reference to the expression of a nucleic acid such as a gene refers to any process which results in an increase in production of a gene product. A gene product can be either RNA (including, but not limited to, mRNA) or protein. Accordingly, gene upregulation or activation includes those processes that increase transcription of a gene and/or translation of a mRNA.

Examples of gene upregulation or activation processes that increase transcription include, but are not limited to, those that facilitate formation of a transcription initiation complex, those that increase transcription initiation rate, those that increase transcription elongation rate, those that increase processivity of transcription and those that relieve transcriptional repression (by, for example. blocking the binding of a transcriptional repressor). Gene upregulation or activation can constitute, for example, inhibition of repression as well as stimulation of expression above an existing level. Examples of gene upregulation or activation processes that increase translation include those that increase translational initiation, those that increase translational elongation, those that increase translational termination, those that increase ribosome recycling, and those that increase mRNA stability. The level of gene expression, including the level of gene activation or upregulation, can be quantitated utilizing a number of established techniques including, but not limited to, Northern-Blots, RNase protection assays (RPA), nucleic acid probe arrays, quantitative PCR (e.g., the so-called TaqMan assays), dot blot assays and in situ hybridization. In general, gene upregulation or activation comprises any detectable increase in the production of a gene product, preferably an increase in production of a gene product by at least 50 to 100%. In other instances from about 2- to about 5-fold or any integer therebetween, in still other instances between about 5- and about 10-fold or any integer therebetween, sometimes between about 10- and about 20-fold or any integer therebetween, in other instances between about 20- and about 50-fold or any integer therebetween, in yet other instances between about 50- and about 100-fold or any integer therebetween, and in still other instances 100-fold or more. The terms upregulated and gene activation can also mean that the observed activity relative to a baseline level is a statistically significant difference (i.e., increase).

As used herein a “baseline value” generally refers to a value (or ranges of values) against which an experimental or determined value (e.g., one determined for a patient sample as part of a diagnostic or prognostic test) is compared. Thus, in the case of upregulation, the baseline value can be a value for activity or expression for a sample obtained from the same individual at a different time point. In other instances, the baseline value is a value determined for a control cell or individual, or a statistical value (e.g., an average or mean) established for a population of control cells or individuals. In upregulation, the control can be a cell, individual or populations thereof for which levels would not be expected to be upregulated. Thus, for instance, a control individual or control population can include healthy individuals. The population that serves as a control can vary in size, having as few as a single member, but potentially including tens, hundreds, thousands, tens of thousands or more individuals. Where the control is a large population, the baseline value can be a statistical value determined from individual values for each member or a value determined from the control population as an aggregate (e.g., a value measured for a population of cells within a well).

In yet another aspect, provided are methods of treating adipocyspin-mediated conditions or diseases by administering to a subject having such a disease or condition, a therapeutically effective amount of an modulator of adipocyspin function, e.g., agonists (stimulators) and antagonists (inhibitors) of adipocyspin function or gene expression.

Diseases and conditions associated with altered adipocyspin expression or activity include obesity and conditions associated with increased adipocyte mass or fat mass. Diseases or conditions, including chronic diseases, can be treated with modulators of adipocyspin function. Diseases or conditions include, for example, obesity and conditions associated with increased fat mass, which may be treated by adipocyspin and adipocyspin agonists. Such modulators include small molecules agonists and antagonists of adipocyspin function or expression, antisense and ribozyme triplex polynucleotides, gene therapy, and so on. The methods and reagents described herein may be used in treatment of animals such as mammals (e.g., humans, non-human primates, cows, sheep, goats, horses, dogs, cats, rabbits, rats, mice, etc.) or in animal or in vitro (e.g., cell-culture) models of human diseases.

Further provided are therapeutic compositions comprising agonists, antagonists, or ligands of adipocyspin, and methods of treating physiologic or pathologic conditions mediated by adipocyspin, including decreased adipocyspin activity.

Adipocyspin polypeptides, fragments thereof, sense and antisense polypeptides, anti-adipocyspin antibodies or binding fragments thereof, and antagonists or agonists (e.g. small molecule modulators) of adipocyspin activity, can be directly administered under sterile or other appropriate or desired conditions to the host to be treated. However, while it is possible for the active ingredient to be administered alone, it may generally be preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious or undesirably toxic to the patient. For example, the bioactive agent can be complexed with carrier proteins such as ovalbumin or serum albumin prior to their administration in order to enhance stability or pharmacological properties such as half-life. Furthermore; therapeutic formulations can be combined with or used in association with other chemotherapeutic or chemopreventive agents.

Therapeutic formulations can be prepared by methods well known in the art of pharmacy. See, e.g., Gilman et at. (eds.) (1990) Goodman and Gilman's: The Pharmacological Bases of Therapeutics (8th ed.) Pergamon Press; and Remington, The Science of Practice and Pharmacy, 20th Edition. (2001) Mack Publishing Co., Easton, P.a.; Avis et al (eds.) (1993) Pharmaceutical Dosage Forms: Parenteral Medications Dekker, N.Y.; Liebennan et al. (eds.) (1990) Pharmaceutical Dosage Forms: Tablets Dekcker, N.Y.; and Liebennan et al (eds.) (1990) Pharmaceutical Dosage Forms: Disperse Systems Dekker, N.Y.

Depending on the disease to be treated and the subject's condition, compounds described herein may be administered by oral, parenteral (e.g., intramuscular, intraperitoneal, intravenous, ICV) intracisternal injection or infusion, subcutaneous injection, or implant), by inhalation spray, nasal, vaginal, rectal. Sublingual, or topical routes of administration and may be formulated, alone or together) in suitable dosage unit formulations containing conventional non-toxic pharmaceutically acceptable carriers, adjuvants and vehicles appropriate for each route of administration.

Pharmaceutical compositions and methods of treatment may further comprise other therapeutically active compounds as noted herein which are usually or may be applied in the treatment of the above mentioned pathological conditions.

In the treatment or prevention of conditions that require adipocyspin modulation, an appropriate dosage level will generally be about 0.001 to about 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to about 25 mg/kg per day, about 0.05 to about 10 mg/kg per day, or about 0.1 to about 5 mg/kg per day. Within this range the dosage may be about 0.005 to about 0.05, 0.05 to about 0.5 or about 0.5 to about 5 mg/kg per day. For oral administration, the compositions are preferably provided in the form of tablets containing from about 1 to about 1000 or more milligrams of the active ingredient, particularly about 1, 5, 10, 15, 20, 25, 50, 75, 100, 150, 200, 250, 300, 400, 500, 600; 750, 800, 900, or 1000 milligrams of the active ingredient for the symptomatic adjustment of the dosage to the patient to be treated. The compounds may be administered on a regimen of about 1 to about 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination the severity of the particular condition and the host undergoing therapy.

Compounds within the patent can be combined with other compounds having related utilities to prevent and treat inflammatory and immunoregulatory disorders and diseases, including asthma and allergic diseases, as well as autoimmune pathologies such as rheumatoid arthritis and atherosclerosis and those pathologies noted above.

Also provided are methods for detection and quantification of adipocyspin polypeptides and polynucleotides in biological samples. In one embodiment, expression or over-expression of the adipocyspin gene product (e.g., polypeptide or mRNA) is correlated with a disease or condition mediated by, or associated with the adipocyspin.

Biological samples include, but are not limited to, a blood sample, serum, cells (including whole cells, cell fractions, cell extracts, and cultured cells or cell lines), tissues (including tissues obtained by biopsy), body fluids (e.g., urine, sputum, amniotic fluid, synovial fluid)) or from media (from cultured cells or cell lines), and the like. The methods of detecting or quantifying adipocyspin polynucleotides include, but are not limited to, amplification-based assays with signal amplification) hybridization based assays and combination amplification-hybridization assays. For detecting and quantifying adipocyspin polypeptides, an exemplary method is an immunoassay that utilizes an antibody or other binding agents that specifically binds to an adipocyspin polypeptide or epitope, for example, ELISA or RIA assays.

The polymerase chain reaction (PCR), or its variations, is an exemplary amplification-based assay. Examples of techniques for in vitro amplification methods are found in PCR TECHNOLOGY: PRINCIPLES AND APPLICATIONS FOR DNA AMPLIFICATION, H. Erlich, Ed. Freeman Press, New York. NY (1992); PCR PROTOCOLS: A GUIDE TO METHODS AND APPLICATIONS, eds. Innis, Gelfland, Snisky, and White, Academic Press, San Diego, Calif. (1990). Other suitable target amplification methods include, for example, the ligase chain reaction (LCR; e.g., Wu and Wallace, 1989, Genomics 4:560); strand displacement amplification (SDA; e.g., Walker et al, 1992, Proc. Natl. Acad. Sci. U.S.A. 89:392-396); the nucleic acid sequence based amplification NASBA, Cangene, Mississauga, Ontario; e.g., Compton, 1991, Nature 350:91), and the like. One useful variant of PCR is PCR ELISA (e.g., Boehringer Mannheim Cat. No. 1 636 111) in which digoxigenin-dUTP is incorporated into the PCR product. The PCR reaction mixture is denatured and hybridized with a biotin-labelled oligonucleotide designed to anneal to an internal sequence of the PCR product. The hybridization products are immobilized on streptavidin coated plates and detected using anti-digoxigenin antibodies.

A variety of methods for specific DNA and RNA measurement using polynucleotide hybridization techniques are known to those of skill in the art (see Sambrook, supra). Hybridization-based assays refer generally to assays in which a polynucleotide probe is hybridized to a target polynucleotide. Usually the polynucleotide hybridization probes such as those described or referenced herein are entirely or substantially identical to a contiguous sequence of the adipocyspin nucleic acid sequence. Preferably, polynucleotide probes are at least about 10 bases, often at least about 20 bases, and sometimes at least about 200 bases or more in length. Methods of selecting polynucleotide probe sequences for use in polynucleotide hybridization are discussed in Sambrook, supra.

Polynucleotide hybridization formats are known to those skilled in the art. In some formats, at least one of the target and probe is immobilized. The immobilized polynucleotide may be DNA, RNA, or another oligo- or poly-nucleotide, and may comprise natural or non-naturally occurring nucleotides, nucleotide analogues, or backbones. Such assays may be in any of several formats including: Southern, Northern, dot and slot blots, high-density polynucleotide or oligonucleotide arrays (e.g., GeneChips™ Affymetrix), dip sticks, pins, chips, or beads. All of these techniques are well known in the art and are the basis of many commercially available diagnostic kits. Hybridization techniques are generally described in Hames et al., ed., NUCLEIC ACID HYBRIDIZATION, A PRACTICAL APPROACH IRL Press, (1985); Gall and Pardue Proc. Nat'l. Acad. Sci., USA., 63: 378-383 (1969); and John et al., Nature, 223: 582-587 (1969).

In one embodiment, in situ hybridization is used to detect adipocyspin sequences in a sample. In situ hybridization assays are well known and are generally described in Angerer et al., METHODS ENZYMOL., 152: 649-660 (1987) and Ausubel, supra.

In one embodiment, an adipocyspin polynucleotide(s) is detected in a sample using an anti-adipocyspin antibody or binding molecule. A number of well established immunological binding assay are suitable for detecting and quantifying adipocyspin. See, e.g., U.S. Pat. Nos. 4,366,241; 4,376,110; 4,517,288; and 4,837,168, and also METHODS IN CELL BIOLOGY VOLUME 37: ANTIBODIES IN CELL BIOLOGY, Asai, ed. Academic Press, Inc. New York (1993); BASIC AND CLINICAL IMMUNOLOGY 7th Edition, Stites & Terr, eds. (1991); Harlow, supra [e.g., Chapter 14], and Ausubel, supra, [e.g., Chapter 11], each of which is incorporated by reference in its entirety and for all purposes.

Immunoassays for detecting adipocyspin may be competitive or noncompetitive. The adipocyspin gene product being assayed may be detected directly or indirectly using a detectable label. The particular label or detectable group used in the assay is usually not a critical aspect, so long as it does not significantly interfere with the specific binding of the antibody or antibodies used in the assay. The label may, for example, be covalently attached to the capture agent (e.g., an anti-adipocyspin antibody), or may be attached to a third moiety, such as another antibody, that specifically binds to the adipocyspin polypeptide at a different epitope than recognised by the capture agent.

Noncompetitive immunoassays are assays in which the amount of captured analyte (here, for example, an adipocyspin polypeptide) is directly measured. One such assay is, for example, a two-site, monoclonal-based immunoassay utilizing monoclonal antibodies reactive to two non-interfering epitopes on the captured analyte. See, e.g., Maddox et al., 1983, J: Exp. Med., 158:1211 for background information. 1 h such an assay, the amount of adipocyspin in the sample is directly measured. For example, using a so-called “sandwich” assay, the capture agent (here, the anti-adipocyspin antibodies) can be bound directly to a solid substrate where they are immobilized.

Immobilized antibodies capture polypeptide present in the test sample. Adipocyspin or other target thus immobilized is then bound by a labelling agent, such as a second adipocyspin antibody bearing a label. Alternatively, for example, the second adipocyspin antibody may lack a label, but it may, in turn, be bound by a labelled third antibody specific to antibodies of the species from which the second antibody is derived. The second can be modified with a detectable moiety, such as biotin, to which a third labelled molecule can specifically bind, such as enzyme-labelled streptavidin.

In competitive assays, the amount of adipocyspin polypeptide present in the sample may be measured indirectly by measuring, for example, the amount of an added (exogenous) adipocyspin displaced (or competed away) from a capture agent (e.g., anti-adipocyspin antibody) by the analyte present in the sample (e.g., adipocyspin polypeptide). In one competitive assay, for example, a known amount of adipocyspin is added to the sample and the sample is then contacted with a capture agent (e.g., an anti-adipocyspin antibody) that specifically binds to adipocyspin. The amount of adipocyspin bound to the antibody is inversely proportional to the concentration of adipocyspin present in the sample.

Preferably, the antibody may be immobilized on a solid substrate. The amount of adipocyspin bound to the antibody may be determined, for example, either by measuring the amount of adipocyspin present in an adipocyspin/antibody complex, or alternatively by measuring the amount of remaining uncomplexed adipocyspin. The amount of adipocyspin may be detected by providing a labelled adipocyspin molecule. For example, using the hapten inhibition assay, the analyte (in this case adipocyspin) is immobilized on a solid substrate. A known amount of anti-adipocyspin antibody, for example, is added to the sample, and the sample is then contacted with the immobilized adipocyspin. In this case, the amount of anti-adipocyspin antibody bound to the immobilized adipocyspin is inversely proportional to the amount of adipocyspin present in the sample. Again the amount of immobilized antibody may be detected, for example, by detecting either the immobilized fraction of antibody or the fraction of the antibody that remains in solution. Detection may be direct where the antibody is labelled or indirect by the subsequent addition of a labelled moiety that specifically binds to the antibody as described above.

In addition to competitive and non-competitive adipocyspin polypeptide immunoassays, also provided are other assays for detection and quantification of adipocyspin polypeptides. For example, Western blot (immunoblot) analysis can be used to detect and quantify the presence of adipocyspin in the sample. The technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with the antibodies that specifically bind adipocyspin. The anti-adipocyspin antibodies specifically bind to adipocyspin on the solid support. These antibodies may be directly labeled or alternatively maybe subsequently detected using labeled antibodies (e.g., labelled sheep anti-mouse antibodies) that specifically bind to the anti-adipocyspin.

Furthermore, assays such as liposome immunoassays (LIA) are also encompassed by the present patent. LIA utilizes liposomes that are designed to bind specific molecules (e.g., antibodies) and to release encapsulated reagents or markers. The released chemicals are then detected according to standard techniques (see, Monroe et al.; 1986, Amer. Clin. Prod. Rev. 5:34-41).

Reagents useful for the therapeutic and diagnostic (detection) methods are conveniently provided in kit form, including kits that contain polypeptides, antibodies, and polynucleotides.

In one embodiment, the kit comprises one or more of the following in a container: (1) one or more adipocyspin polynucleotides (e.g., oligonucleotide primers or probes corresponding to the adipocyspin cDNA sequence and capable of amplifying the target polynucleotides); (2) one or more anti-adipocyspin antibodies (or other binding molecules); (3) one or more adipocyspin polypeptides or fragments, optionally coated on, for example, a solid surface (such as a slide, multiple well plate, or test tube) (4) one or more adipocyspin polynucleotides and/or polypeptides (e.g., for use as positive controls in assays), (5) and tubes. Instructions for carrying out relevant detection methods and, for example, calibration curves can also be included.

In the following study, we report the identification of a novel adipocyte-secreted product which shares sequence homology with family members of cysteine protease inhibitors. The expression of adipocyspin was induced markedly during adipose conversion of 3T3-L1 cells. The mRNA abundance was also significantly increased in obesity states. Furthermore, treatment of 3T3 L1 cells with recombinant adipocyspin significantly inhibited the adipose conversion, indicating that adipocyspin is a negative regulator of adipogenesis.

A further understanding will be gained by reference to the following experimental section the following experiments are illustrative and are not intended to limit the invention in any way.

### EXAMPLE 1

Sets out the experimental procedures utilised to differentiate 3T3-L1 cells and subsequent concentration of the resultant protein from the cell culture medium.

3T3-L1 cells were maintained as subconfluent cultures in DMEM that was supplemented with 10% fetal calf serum. For differentiation to occur, the cells were seeded onto 150 mm plates and allowed to reach 100% confluence and induced one-day post confluence with the above medium containing 0.25 μM dexamethasone, 0.5 mM IBMX and 10 μg/ml insulin for 2 days. This was then followed by incubation with 10 μg/ml insulin for 2 days. The cells were then maintained in DMEM with 10% fetal calf serum for another 4 days.

To harvest the proteins secreted from the adipocytes, the cells 8 days after differentiation, were washed three times with PBS, and then incubated with serum-free medium for another 4 hours. The medium was then collected, centrifuged at 3,000×g for 10 min, filtered through 0.20 μm filter, and then concentrated and desalted using a concentrator with MWCO of 5000 Da (Vivascience Ltd, Gloucestershire, UK). The proteins were then quantitated using BCA reagent, and stored at −80° C. until use.

The proteins secreted from either adipocytes or 3T3 L1 preadipocytes were separated by two dimensional gel electrophoresis as described previously, using Immobiline DryStrips with a pH range of 6-11. The separated proteins were stained with either silver or Coomassie Brilliant Blue R250 (CBB). The differentially secreted proteins were identified by Melanine 2 software.

Proteins of interest that were separated by 2-DE gels were excised and gel pieces were subjected to in-gel trypsin digestion as described previously. The extracted tryptic peptide mixtures were fractionated by RP HPLC on a Jupiter 5μ C18 column (250×2.00 mm, Phenomenex). The pre-warmed column (37° C.) was washed for 7 min with 0.1% trifluoroacetic acid (v/v) followed by elution using a 50 min linear gradient from 8% to 36% of acetonitrile at the flow rate of 200 μl/min. The well-separated fractions were chosen for amino acid sequencing using the Edman degradation method with a Perkin-Elmer (Procise, Model 492) protein sequencer.

Cloning and mammalian expression of adipocyspin. Total RNA was purified from mouse 3T3-L1 adipocytes or human fat pads using TRIZOL reagent according to the manufacturer's instructions. The oligo-dT-primed cDNA from the total RNA was used as a template for PCR cloning. The full-length cDNAs of mouse (SEQ ID NO: 5) and human (SEQ ID NO: 6) adipocyspin were inserted into pGEMT-easy vector (Promega) for DNA sequence verification.

The vector for mammalian expression of mouse adipocyspin was generated by cDNA amplification using 5′GCCCGCGGATCCATGCTACTGTTGCAAGCTCT3′ [SEQ ID NO: 3] as the sense primer and 5′GGCCGCGAATTCTCACTTGTCATCGTCGTCCTTGTAGTCGTTGGTATCATGGTAG AG3′ [SEQ ID NO.: 4] as the antisense primer. Following digestion with BamHI/EcoRI, the fragment was inserted into pcDNA3.1 vector to produce pcDNA-Adipocyspin-F, which encodes full-length adipocyspin with a FLAG epitope tag at its C-terminus. This mammalian expression vector was transfected into COS-7 cells using FuGENE 6 transfection reagent, and the cells were allowed to secrete adipocyspin into serum free-medium for 48 hr. The medium was then harvested and the cell debris removed by centrifugation at 3,000×g for 10 min followed by filtration through a 0.2 μm filter. The filtered medium was concentrated using a Vivian concentrator with MWCO of 5000 Da, as described above. FLAG tagged adipocyspin was purified using anti-FLAG M2 affinity gel and eluted with 150 μg/ml of FLAG peptide, per manufacturer's instructions (Sigma).

Northern blot and Western blot analysis. 10 μg of total RNA purified from either 3T3 L1 cells or mouse adipose tissue was separated on a 1.2% formaldehyde-denaturing agarose gel and transferred to Nylon membranes. Hybridization was carried out as described previously; using ³²P labelled full-length adipocyspin, adiponectin, PPAR γ, or GLUT4 cDNAs as a probe. The membranes were visualized and analysed using a phosphorimager. Western blot analysis was performed as described previously.

### EXAMPLE 2

The following experiment sets out the characterization of adipocyspin.

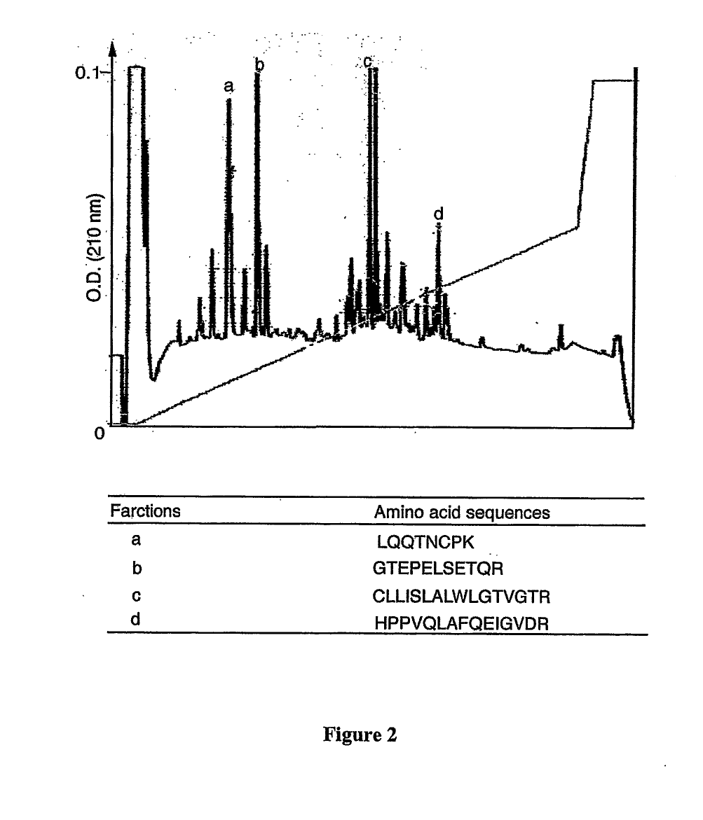

The proteins from culture medium of 3T3 L1 preadipocytes and adipocytes were separated by two-dimensional gel electrophoresis. Analysis showed that a protein with apparent MW of 16 kDa and pI value of 9.3 was preferentially present in the adipocytes and not in preadipocytes (FIG. 1). To identify the nature of this protein, the protein “spots” (see FIG. 1) were excised from multiple preparative gels and then subjected to in-gel trypsin digestion. The tryptic peptide mixtures were fractionated by RP HPLC and the well-separated fractions were subjected to amino acid sequencing (FIG. 2). The amino acid sequences derived from the four tryptic peptides could not be assigned to any known proteins. tBLASTn searching of the nucleic acid database at the National Center for Biotechnology Information indicated a match with a hypothetical protein electronically translated from an expressed sequence tag (EST) sequence from RIKEN full-length enriched murine adult cDNA library (gene accession number: AK002298). RT PCR analysis confirmed expression of the gene in 3T3 L1 adipocytes.

The hypothetical reading frame of this cDNA sequence encodes a putative protein of 162 amino acid residues (FIG. 3A). The predicted amino acid sequence contains cysteine residues at amino acid positions 62, 72, 83, 86, 101 and 116, as shown in FIG. 3A. One cysteine residue may be joined to form an intramolecular disulphide bond with another cysteine residue, such that adipocyspin and/or adipocyspin isoforms may contain up to 3 intramolecular disulphide bonds. One hydrophobic stretch predicted following Kyte-Doolittle plot analysis is located within the first 17 amino-terminal residues and is characteristic of a signal sequence. Homology searching revealed some similarity between the N-terminal half of this protein and a family of proteins with cystatin-like domain such as cystatin C (FIG. 3B). Cystatins are a family of cysteine protease inhibitors and many of them are secretory proteins. The COOH-terminal half of adipocyspin displays little homology to any known proteins. The predicted molecular mass and pI value of adipocyspin (excluding the putative secretory signal) is 16548.23 Da and 9.36, which perfectly matches tQ the values observed during 2DE separation (FIG. 2).

### EXAMPLE 3

This experiment confirms adipocyspin as a secretory protein. A FLAG epitope tagged adipocyspin construct was introduced into COS 7 cells by transient transfection and the protein was detected in the conditioned medium by immunoblotting.

Analysis showed that adipocyspin can be readily detected in the culture medium (FIG. 4). On the other hand, β tubulin, a cytoplasmic protein, was hardly detectable, and it was concluded that adipocyspin in the cell culture medium is not due to cell lysis.

### EXAMPLE 4

This example assessed the differentiation-dependent expression of adipocyspin mRNA by examining the time course of adipocyspin mRNA expression during the adipose conversion of 3T3 L1 preadipocytes by Northern blot analysis. As shown in FIG. 5, adipocyspin mRNA expression correlates well with cell differentiation and changes in cell morphology (round shape and appearance of intracellular lipid droplets). Adipocyspin mRNA having approximately 800 bp started to appear as early as day 2 following induction of adipose conversion, and reached a maximum at day 8. The expression kinetics of adipocyspin paralleled those of aP2, and slightly preceeded adiponectin, a protein exclusively expressed in adipocytes.

Results demonstrated a simultaneous appearance of adipocyspin mRNA expression and adipocyte phenotype.

### EXAMPLE 5

Work in this example assessed the altered expression of adipocyspin in obesity states. Modulation of gene expression in obesity provides valuable information for functional relevance of the protein of interest in metabolic disease states.

We observed a consistent three to four-fold increase of adipocyspin mRNA in obese (ob/ob) mice relative to their cogenic lean controls (FIG. 6). This result is in sharp contrast with the decreased expression of adiponectin gene in ob/ob mice. Altered expression of adipocyspin in obesity indicates involvement of this protein in one or more pathophysiological features of such states. Adipocyspin mRNA levels may be controlled by genetic defects specific for this model, e.g., leptin.

### EXAMPLE 6

Work in this example assessed adipocyspin inhibition of adipocyte differentiation proteolysis and its role in adipose conversion. The involvement of adipocyspin in adipocyte differentiation was tested.

COOH-terminal FLAG-tagged adipocyspin was purified from the culture medium of transiently transfected COS 7 cells and then added to 3T3 L1 cells. In the absence of adipocyspin, over 80% of 3T3 L1 cells were differentiated into lipid-laden adipocytes, as shown in oil red O staining (see A of FIG. 7). In the cells treated with 20 μg/ml adipocyspin, only sporadic occurrence of lipid laden cells (less than 1 in 50) was observed. Similarly, the expression of adipocyte markers, PPAR γ and GLUT4, was also decreased by over 70% when the cells were treated with adipocyspin (FIG. 8). These results indicate that adipocyspin can block adipose conversion.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The inventions illustratively described herein suitably may be practiced in the absence of any element or elements, or limitation or limitations, which is not specifically disclosed herein as essential. Thus, for example, in each instance herein, and descriptions of embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention may be specifically or generally disclosed by various embodiments and/or preferred embodiments and optional features, any and all modifications and variations of the concepts herein disclosed that can or may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material or the proviso is specifically recited herein.

It is also to be understood that as used herein and in the appended claims, the singular forms “a,” “an,” and “the” include plural reference unless the context clearly dictates otherwise, and the letter “s” following a noun designates both the plural and singular forms of that noun. The term “or” when used herein as the sole conjunction means “and/or” unless stated otherwise. The term “including” and related terms such as “includes” as used herein are not limiting and allow for the presence of elements in addition to those specifically recited. In addition, where features or aspects are described in terms of Markush groups, those skilled in the art will recognize that the invention is also thereby described in terms of any individual member or subgroup of members of the Markush group.

Other embodiments are within the following claims. Under no circumstances may the patent be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1-30. (canceled)

31. An isolated polynucleotide which encodes an adipocyspin polypeptide.

32. A recombinant polynucleotide according to claim 31.

33. A polynucleotide according to claim 31 which is substantially pure.

34. A polynucleotide according to claim 31 which encodes a human adipocyspin polypeptide.

35. A polynucleotide according to claim 34 which comprises SEQ ID NO: 6.

36. A polynucleotide according to claim 34 comprising a nucleotide sequence which encodes SEQ ID NO: 2.

37. A polynucleotide according to claim 31 which encodes a rat adipocyspin, a mouse adipocyspin or a chicken adipocyspin.

38. A polynucleotide according to claim 37 comprising a nucleotide sequence which comprises SEQ ID NO: 5, SEQ ID NO: 7 or SEQ ID NO: 8.

39. A polynucleotide according to claim 37 which comprises a nucleotide sequence encoding SEQ ID NO: 1, SEQ ID NO: 9 or SEQ ID NO: 10.

40. A polynucleotide which encodes an adipocyspin polypeptide variant and wherein said polynucleotide is able to hybridize to a polynucleotide of claim 31 under stringent conditions.

41. A polynucleotide which encodes an adipocyspin polypeptide variant and wherein said polynucleotide is able to hydridize to a polynucleotide of claim 31 having a nucleotide sequence comprising SEQ ID NO: 5, SEQ ID NO: 6, SEQ ID NO: 7 or SEQ ID NO: 8 under stringent conditions.

42. An expression vector comprising a polynucleotide of any of claims 31, 34 to 40 or 41.

43. A cell comprising a vector of claim 42.

44. A cell according to claim 43 which is an eukaryotic cell.

45. A cell according to claim 44 which is a mammalian cell.

46. A cell according to claim 43 which is a bacterial cell.

47. An isolated adipocyspin polypeptide.

48. A polypeptide according to claim 47 which is a recombinant polypeptide.

49. A polypeptide according to claim 47 which is substantially pure.

50. A polypeptide according to claim 49 wherein the polypeptide is more than 80% pure.

51. A polypeptide according to claim 49 wherein the polypeptide is more than about 90% pure.

52. A polypeptide according to claim 49 wherein the polypeptide is more than about 95% pure.

53. A polypeptide according to claim 47 which comprises a human adipocyspin polypeptide.

54. A polypeptide according to claim 53 which comprises SEQ ID NO: 2.

55. An adipocyspin polypeptide variant having adipocyspin activity which comprises an amino acid sequence at least about 60% identical to SEQ ID NO: 2.

56. An adipocyspin polypeptide fragment having adipocyspin activity which comprises an amino acid sequence at least about 60% identical to SEQ ID NO: 2.

57. A polypeptide according to claim 47 which comprises a rat adipocyspin, a mouse adipocyspin or a chicken adipocyspin.

58. A polypeptide according to claim 57 which comprises SEQ ID NO: 1, SEQ ID NO: 9 or SEQ ID NO: 10.

59. An adipocyspin polypeptide variant having adipocyspin activity and which comprises an amino acid sequence which is at least about 60% identical to SEQ ID NO: 1, SEQ ID NO: 9 or SEQ ID NO: 10.

60. An adipocyspin fragment having adipocyspin activity and which comprises an amino acid sequence which is at least about 60% identical to SEQ ID NO: 1, SEQ ID NO: 9 or SEQ ID NO: 10.

61. A composition comprising a therapeutically effective amount of an adipocyspin polypeptide according to claim 47 and a pharmaceutically acceptable carrier.

62. A composition according to claim 61 wherein the adipocyspin polypeptide is recombinant.

63. A composition according to claim 62 wherein the adipocyspin polypeptide is substantially pure.

64. A composition according to claim 63 wherein the adipocyspin polypeptide is more that 80% pure.

65. A composition according to claim 63 wherein the adipocyspin polypeptide is more than about 90% pure.

66. A composition according to claim 63 wherein the adipocyspin polypeptide is more than about 95% pure.

67. A composition according to claim 61 wherein said adipocyspin polypeptide is a human adipocyspin.

68. A method for decreasing adipose tissue mass in a subject which comprises administering to said subject an effective amount of an adipocyspin polypeptide of claim 47.

69. A method according to claim 68 wherein said subject is a human.

70. A method according to claim 68 wherein the adipocyspin polypeptide is administered in an amount effective to elicit a plasma adipocyspin polypeptide concentration between 1 μg/mL and 20 μg/mL.

71. A method according to claim 68 wherein the adipocyspin polypeptide is administered in an amount effective to elicit a plasma polypeptide concentration of between 1.9 μg/mL and 17 μg/mL.
